(19) United States
(12) Patent Application Publication
(10) Pub. No.: US 2024/0354940 A1
(43) Pub. Date: Oct. 24, 2024

(54) SYSTEMS AND METHODS FOR FACILITATING LESION INSPECTION AND ANALYSIS

(71) Applicants: Progenics Pharmaceuticals, Inc., N. Billerica, MA (US); EXINI Diagnostics AB, Lund (SE)

(72) Inventors: Karl Vilhelm Sjöstrand, Atlantic Highlands, NJ (US); Jens Filip Andreas Richter, Staffanstorp (SE); Johan Martin Brynolfsson, Helsingborg (SE); Hannicka Maria Eleonora Sahlstedt, Malmö (SE)

(21) Appl. No.: 18/627,691

(22) Filed: Apr. 5, 2024

Related U.S. Application Data
(60) Provisional application No. 63/606,803, filed on Dec. 6, 2023.
(60) Provisional application No. 63/457,974, filed on Apr. 7, 2023.

(51) Int. Cl. G06T 7/00; G06F 3/04842; G06V 10/25; G06V 10/40; G06V 10/70

(52) U.S. Cl. CPC G06T 7/0012; G06F 3/04842; G06V 10/25; G06V 10/40; G06V 10/70; G06T 2207/30096

(57) ABSTRACT

Presented herein are systems, methods, and architectures related to the identification and presentation of hotspots (e.g., cancerous regions (e.g., metastatic) and/or regions suspected of being cancerous, e.g., 3D regions) in medical images. In certain embodiments, a slider and/or other graphical user interface widget is provided to allow intuitive, interactive adjustment by a user for inclusion and/or exclusion of hotspots (e.g., thresholds or other criteria for selection of a hotspot or other ROI are adjusted by the user by manipulation of the slider or other GUI widget).

### CROSS REFERENCE TO RELATED APPLICATIONS

This application claims priority to and benefit of U.S. Provisional Patent Application No. 63/606,803, filed Dec. 6, 2023, and U.S. Provisional Patent Application No. 63/457,974, filed Apr. 7, 2023, the contents of each of which are incorporated by reference herein in their entirety.

### FIELD

This invention relates generally to systems and methods for analysis of medical images. More particularly, in certain embodiments, the present disclosures provides systems and methods for graphical control of medical images and/or regions of interest identified therein.

### BACKGROUND

Nuclear medicine imaging involves the use of radiolabeled compounds, referred to as radiopharmaceuticals. Radiopharmaceuticals are administered to patients and accumulate in various regions in the body in manner that depends on, and is therefore indicative of, biophysical and/or biochemical properties of tissue therein, such as those influenced by presence and/or state of disease, such as cancer. For example, certain radiopharmaceuticals, following administration to a patient, accumulate in regions of abnormal osteogenesis associated with malignant bone lesions, which are indicative of metastases. Other radiopharmaceuticals may bind to specific receptors, enzymes, and proteins in the body that are altered during evolution of disease. After administration to a patient, these molecules circulate in the blood until they find their intended target. The bound radiopharmaceutical remains at the site of disease, while the rest of the agent clears from the body.

Nuclear medicine imaging techniques capture images by detecting radiation emitted from the radioactive portion of the radiopharmaceutical. The accumulated radiopharmaceutical serves as a beacon so that an image may be obtained depicting the disease location and concentration using commonly available nuclear medicine modalities. Examples of nuclear medicine imaging modalities include bone scan imaging (also referred to as scintigraphy), single-photon emission computerized tomography (SPECT), and positron emission tomography (PET). Bone scan, SPECT, and PET imaging systems are found in most hospitals throughout the world. Choice of a particular imaging modality depends on and/or dictates the particular radiopharmaceutical used. For example, technetium 99m (^{99m}Tc) labeled compounds are compatible with bone scan imaging and SPECT imaging, while PET imaging often uses fluorinated compounds labeled with 18F. The compound ^{99m}Tc methylenediphosphonate (^{99m}Tc MDP) is a popular radiopharmaceutical used for bone scan imaging in order to detect metastatic cancer. Radiolabeled prostate-specific membrane antigen (PSMA) targeting compounds such as ^{99m}Tc labeled 1404 and PyL™ (also referred to as [18F]DCFPyL) can be used with SPECT and PET imaging, respectively, and offer the potential for highly specific prostate cancer detection.

Accordingly, nuclear medicine imaging is a valuable technique for providing physicians with information that can be used to determine the presence and the extent of disease in a patient. The physician can use this information to provide a recommended course of treatment to the patient and to track the progression of disease.

For example, an oncologist may use nuclear medicine images from a study of a patient as input in her assessment of whether the patient has a particular disease, e.g., prostate cancer, what stage of the disease is evident, what the recommended course of treatment (if any) would be, whether surgical intervention is indicated, and likely prognosis. The oncologist may use a radiologist report in this assessment. A radiologist report is a technical evaluation of the nuclear medicine images prepared by a radiologist for a physician who requested the imaging study and includes, for example, the type of study performed, the clinical history, a comparison between images, the technique used to perform the study, the radiologist's observations and findings, as well as overall impressions and recommendations the radiologist may have based on the imaging study results. A signed radiologist report is sent to the physician ordering the study for the physician's review, followed by a discussion between the physician and patient about the results and recommendations for treatment.

Moreover, computed tomography (CT) scans can be performed to identify and display detailed images of specific anatomical regions (e.g., organs and/or tissue). CT scans (e.g., three-dimensional (3D) scans), conventional X-rays, magnetic resonance imaging (MRI), or other scans that provide anatomical imaging can be used with the above-described functional nuclear medicine imaging—e.g., bone scan (scintigraphy), PET, and/or SPECT imaging, to provide anatomical context, e.g., to identify bones, organs, and/or other tissues in which metastases identified via functional imaging are located.

A disease scanning process may involve having a radiologist perform an imaging study on the patient, analyzing the images obtained, creating a radiologist report, forwarding the report to the requesting physician, having the physician formulate an assessment and treatment recommendation, and having the physician communicate the results, recommendations, and risks to the patient. The process may also involve repeating the imaging study due to inconclusive results, or ordering further tests based on initial results. If an imaging study shows that the patient has a particular disease or condition (e.g., cancer), the physician discusses various treatment options, including surgery, as well as risks of doing nothing or adopting a watchful waiting or active surveillance approach, rather than having surgery.

Accordingly, the process of reviewing and analyzing multiple patient images, over time, plays a critical role in the diagnosis and treatment of cancer, for example, identifying the progression, arrest, or diminishment of cancer in the patient over time as a result of treatment. There is, thus, a significant need for improved tools that facilitate and improve accuracy of image review and analysis for cancer diagnosis and treatment. Improving the toolkit utilized by physicians, radiologists, and other healthcare professionals in this manner provides for significant improvements in standard of care and patient experience.

### SUMMARY

Presented herein are systems, methods, and architectures related to the identification and presentation of hotspots (e.g., cancerous regions (e.g., metastatic) and/or regions suspected of being cancerous, e.g., 3D regions) in medical images. In certain embodiments, a slider and/or other graphical user interface widget is provided to allow intuitive, interactive adjustment by a user for inclusion and/or exclusion of hotspots (e.g., thresholds or other criteria for selection of a hotspot or other ROI are adjusted by the user by manipulation of the slider or other GUI widget).

Upon automated or semi-automated detection and selection of such hotspots, analyses can be run, for example, to determine overall hotspot volume and/or to determine various disease measurement or risk indices, wherein said indices may be weighted by location of the identified hotspots. Among other things, the present disclosure includes the insight that, on the one hand, in cases where disease burden is high and a large number of hotspots have been detected, one-at-a-time selection of hotspots can be tedious and time consuming for a user. On the other hand, selecting all hotspots detected in an entire image and/or within particular miTNM classifications can lead to the selection of an excessively large number of hotspots and inclusion of undesired hotspots in a user-selected set. Certain detection approaches, such as machine learning models, that automatically detect hotspots are often tuned to high sensitivity, so as to be over inclusive, sacrificing positive predictive value—this may be done, for example, especially in the context of medical imaging and cancer detection since a real (medical/human) cost of a false negative (e.g., not detecting a cancerous lesion) can far outweigh that of a false positive.

Accordingly, in recognition of these aforementioned challenges, systems and methods of the present disclosure provide convenient and user-friendly graphical tools that allow a user to rapidly select hotspots yet, at the same time, maintain fine control over which hotspots are included, even when a large number of hotspots have been detected in high-disease burden cases. As described herein, the selected hotspots may be used to determine overall hotspot volume and/or to determine various disease measurement or risk indices, wherein said indices may be weighted by location of the identified hotspots.

Additionally or alternatively, as described herein, the present disclosure introduces semi-automated, interactive graphical tools allowing a user to efficiently and intuitively edit and fine tune segmented hotspot volumes, edit anatomical location assignments, and identify and segment hotspots via a “single-click.” Thus, among other things, graphical control tools of the present disclosure allow a user to conveniently review and update medical images and lesion information determined therefrom according to their expert analysis in an efficient, rapid, and convenient fashion.

In one aspect, the invention is directed to a method for interactive control of selection and/or analysis of hotspots (e.g., regions of cancer and/or regions suspected of being cancer, e.g., metastases) detected within a medical image [e.g., a three-dimensional image, e.g., a nuclear medicine image (e.g., bone scan (scintigraphy), PET, and/or SPECT), e.g., an anatomical image {e.g., computed tomography (CT), X-ray, or magnetic resonance imaging (MRI) scan}, e.g., a combined nuclear medicine and anatomical image (e.g., overlaid)] of a subject and representing potential lesions, the method comprising: (a) receiving and/or accessing, by a processor of a computing device, (i) an identification of a plurality of hotspots having been detected within the medical image using a machine learning model, and, (ii) a set of hotspot feature values comprising, for each particular hotspot of the plurality of detected hotspots, a corresponding value of at least one hotspot feature representing and/or indicative of a certainty or confidence in detection of the particular hotspot (e.g., a measure/determination of intensity, size, lesion type, and/or other graphical feature or combination of features of the particular hotspot) and/or a likelihood that the particular hotspot represents a true physical lesion within the subject [e.g., a corresponding likelihood value having been determined (e.g., as output) by the machine learning model upon and/or together with detection of the particular hotspot]; (b) causing, by the processor, display of a graphical control element allowing for user selection of a subset of the plurality of detected hotspots via user adjustment of one or more displayed indicator widgets within the graphical control element from which values of one or more criteria (e.g., thresholds) are determined; (c) determining, by the processor, based on a user adjustment of [e.g., a position (e.g., of a slider along a scale) of; e.g., a status (e.g., of a checkbox, radio button, etc.) of; e.g., an entry or selection of alphanumeric character(s) within (e.g., wherein the indicator widget comprises a text box, drop down menu, or the like)] the one or more displayed indicator widgets, user selected values of the one or more criteria (e.g., thresholds); (d) selecting, by the processor, a user-selected subset of the plurality of detected hotspots based on (i) the set of hotspot feature values and (ii) the user selected values of the one or more criteria (e.g., thresholds); and (e) storing and/or providing (e.g., rendering), by the processor, for display and/or further processing, an identification of the user-selected subset.

In certain embodiments, a particular one of the one or more criteria is a rank threshold whose value corresponds to a position on an ordered (e.g., ranked) list, and the method comprises: at step (c), determining the value of the rank threshold; and at step (d), ordering the plurality of hotspots according to their corresponding feature values in the set of hotspot feature values, thereby creating an ordered list of hotspots and selecting, as the user-selected subset, those hotspots having a position in the ordered list of hotspots above and/or below the value of the rank threshold.

In certain embodiments, the at least one hotspot feature is or comprises one or more of (i) to (iii) as follows: (i) a hotspot size (e.g., volume) that provides a measure of size of a particular hotspot, (ii) a hotspot intensity (e.g., voxel intensity value) that provides a measure of intensity a particular hotspot, and (iii) an intensity-weighted hotspot size, providing a measure (e.g., and determined as a function) of both size and intensity of a particular hotspot.

In certain embodiments, the at least one hotspot feature is or comprises a lesion classification (e.g., miTNM classification) that classifies a given hotspot according to a particular lesion labeling and classification scheme (e.g., an miTNM classification scheme).

In certain embodiments, the at least one hotspot feature is or comprises a lesion location identifying an anatomical location of an underlying physical lesion that a given hotspot represents [e.g., wherein the anatomical location is a tissue region or organ selected from a (e.g., pre-defined) set of locations {e.g., wherein at least one member of the set of locations is a skeletal region identifying hotspots representing lesions within bone of the subject; e.g., wherein at least one member of the set of locations is a prostate region identifying hotspots representing lesions with a prostate of the subject; e.g., wherein at least one member of the set of locations is a lymph region, identifying hotspots representing lesions within lymph nodes of the subject; e.g., wherein one or more members of the set of locations is/are one or more particular lymph node regions and/or lymph sub-regions (e.g., pelvic lymph) identifying hotspots representing lesions within particular lymph nodes and/or lymph sub-regions (e.g., pelvic lymph) within the subject; e.g., wherein one or more members of the set of locations is a liver region identifying hotspots representing lesions within a liver of the subject; e.g., wherein one or more members of the set of locations is an arm region identifying hotspots representing lesions within one or both arms (e.g., at least a portion of one or both humerus bones) the subject; e.g., wherein one or more members of the set of locations is a skull region identifying hotspots representing lesions within a skull or head of the subject}].

In certain embodiments, the at least one hotspot feature is or comprises a likelihood value having been determined (e.g., as output) by the machine learning model upon and/or together with detection of a given hotspot and representing a likelihood, as determined by the machine learning model, that the given hotspot represents a true physical lesion within the subject.

In certain embodiments, the one or more criteria [e.g., specified by the user via adjustment of the one or more displayed indicator widgets, e.g., via user entry of alphanumeric character(s) within a textbox of the displayed widget(s) and/or via user selection of alphanumeric characters presented to the user via a drop-down menu of the displayed widget(s)] comprises one or more of (i)-(iii) as follows: (i) tumor/lesion type classification [e.g., miTNM classification, e.g., local tumors (T), regional nodes (N), and/or distant metastases (M)] [e.g., such that user adjustment of the one or more displayed indicator widgets causes selection of all hotspots having a particular user-specified miTNM classification (e.g., the particular user-specified miTNM classification being the user-selected value of the tumor/lesion type classification criteria)], (ii) a measure of hotspot intensity [e.g., such as a standardized uptake value (SUV) (e.g., such that user adjustment of the one or more displayed indicator widgets causes selection of all hotspots exceeding a user-identified minimum threshold SUV value); e.g., where SUV reflects intensity of the hotspot in relation to a reference intensity level], and (iii) a measure of volume (e.g., such that user adjustment of the one or more displayed indicator widgets causes selection of all hotspots exceeding a user-identified minimum volume, e.g., a normalized volume).

In certain embodiments, the one or more criteria comprise a hotspot likelihood threshold [e.g., such that user adjustment of the one or more displayed indicator widgets causes selection of all hotspots having corresponding (e.g., machine-learning model-determined) likelihood values that are equal to and/or exceed the hotspot likelihood value].

In certain embodiments, the method comprises causing, by the processor, graphical rendering of one or both of (i) the plurality of detected hotspots and/or (ii) the user-selected subset of the plurality of detected hotspots, wherein each hotspot is rendered as a (e.g., colorized) graphical shape and/or outline thereof (e.g., identifying/demarcating the hotspot) overlaid on the medical image.

In certain embodiments, the method comprises causing, by the processor, rendering a plurality of graphical shapes as overlaid on the medical image, each of the plurality of graphical shapes corresponding to and demarking a detected hotspot and having a solid, partially transparent, fill (e.g., colored fill); receiving, by the processor, via a user interaction with an opacity setting graphical widget, an opacity value (e.g., ranging from totally opaque to totally transparent); and updating, by the processor, an opacity of the solid fill and/or boundary of the graphical shapes according to the user-selected opacity value.

In certain embodiments, the method comprises causing, the processor, rendering, for each detected hotspot, a graphical outline demarcating a boundary of the hotspot overlaid on the medical image [e.g., the graphical outline is unfilled, such that voxels of the medical image within the outline are unobscured; e.g., wherein the graphical outline is rendered to (e.g., directly) follow boundaries of individual voxels of the medical image; e.g., wherein the rendered outline is smoothed] {e.g., wherein the method comprises receiving a user-selected outline style (e.g., filled area with outline, filled area only, outline only, outline follows boundaries of individual pixels of the medical image, outline follows a smoothed curve of individual pixels of the medical image) and causing rendering of the detected hotspots according to the user-selected outline style}.

In certain embodiments, the method comprises receiving, by the processor, a user selection of a particular hanging protocol (e.g., simultaneous display of axial, coronial, and sagittal projections of the medical image) and causing, by the processor, display of the medical image according to the particular (i.e., user-selected) hanging protocol (e.g., within an image viewing and/or analysis graphical user interface (GUI)).

In certain embodiments, the set of hotspot feature values comprises, for each particular hotspot of the plurality of detected hotspot, a lesion location assignment having an initial value: (i) identifying a particular anatomical region in which the particular hotspot (e.g., the potential physical lesion that it represents) is (e.g., determined, by the processor to be) located or (ii) identifying the particular hotspot has as unassigned [e.g., having an indeterminate or as yet-to-be specified location; e.g., not associated with any particular anatomical region (e.g., to be determined)]; the one or more user-selected criteria comprise a lesion location assignment criteria; and the method comprises: at step (c), determining, via the user interaction with the one or more displayed indicator widgets, as the value of the lesion location assignment criteria, an unassigned hotspots value (e.g., thereby causing selection of all unassigned hotspots); at step (d), selecting, by the as the user-selected subset, all unassigned hotspots; causing, by the processor, graphical rendering and display of the user-selected subset; and for each of particular hotspot of at least a portion of the user-selected subset (e.g., the unassigned hotspots): receiving, by the processor, a user input (e.g., selection) of a location assignment for the particular hotspot; and updating the lesion location assignment for the particular hotspot with the user input (e.g., selected) location assignment.

In certain embodiments, the method comprises receiving, by the processor, a user selection of a particular hotspot of the plurality of detected hotspots [e.g., optionally, causing rendering of the user selected hotspot in a manner that visually emphasizes and/or differentiates the user-selected hotspot relative to other (e.g., un-selected) hotspots (e.g., hiding/obscuring/otherwise differentiating from the un-selected hotspots)]; receiving, by the processor a user selection of one or more voxels of the medical image to add to, and/or subtract from the particular (i.e., user-selected) hotspot; and updating the particular (i.e., user-selected) hotspot to incorporate and/or exclude the one or more user-selected voxels.

In certain embodiments, at least a portion of the medical image is rendered and display for user viewing and/or review within a graphical user interface (GUI) and the method comprises: receiving, by the processor, one or more user-identified points (e.g., voxels) within the medical image, each of the one or more user-identified points corresponding to a location of a user single-click within the GUI; for each particular one of the one or more user-identified points within the medical image, segmenting, by the processor, the medical image to delineate a 3D volume of corresponding user-specified hotspot using (i) the particular user-identified point and (ii) intensities of voxels of the medical image about [e.g., in proximity to; e.g., within a sub-volume of the medical image enclosing (e.g., centered on) the particular user-identified point (e.g., the sub-volume defined by one or more threshold distances (e.g., a spherical volume centered on and comprising voxels within a threshold distance/radius from the particular user-identified point; e.g., a rectangular volume with particular x,y,z dimensions centered on the user-identified point)] the particular user-identified point, thereby determining one or more user-specified hotspots, each associated with and segmented via a user single-click; and updating, by the processor, the initial set of hotspots to include the plurality of user-specified hotspots.

In certain embodiments, for each particular one of the one or more user-identified points within the medical image, segmenting the medical image to delineate the 3D volume of the corresponding user-specified hotspot comprises: determining a local intensity threshold value based on the intensities of the voxels of the medical image about the particular user-identified point [e.g., and one or more reference intensity values, each associated with and determined using intensities of voxels within a corresponding reference tissue region (e.g., a liver; e.g., an aorta portion; e.g., parotid)]; and using the local intensity threshold value to segment the medical image to delineate the 3D volume of the corresponding user-specified hotspot.

In certain embodiments, for each particular one of the one or more user-identified points within the medical image, segmenting the medical image to delineate the 3D volume of the corresponding user-specified hotspot comprises: determining an initial intensity threshold value using the particular user-identified point; at a first step, using the initial intensity threshold value to segment the medical image to identify and delineate a connected region within the medical image and determining an updated intensity threshold value based on intensities of the medical image within the connected region; and at a second step (e.g., iteration), segmenting the medical image to identify and delineate an updated connected region updating the intensity threshold value based on intensities of the medical image within the updated connected region.

In certain embodiments, the medical image is or comprises a 3D functional image. In certain embodiments, the medical image is or comprises a nuclear medicine image. In certain embodiments, the medical image is or comprises a positron emission tomography (PET) image and/or a single photon emission computed tomography (SPECT) image obtained following administration of an agent to the subject. In certain embodiments, the agent comprises a PSMA binding agent. In certain embodiments, the medical image is or comprises a PET image. In certain embodiments, the agent comprises ¹⁸F. In certain embodiments, the agent is or comprises [18F]DCFPyL. In certain embodiments, the agent is or comprises PSMA-11. In certain embodiments, the agent comprises one or more members selected from the group consisting of ^{99m}Tc, ⁶⁸Ga, ¹⁷⁷Lu, ²²⁵ Ac, ¹¹¹In, ¹²³I, ¹²⁴I, and ¹³¹I.

In another aspect, the invention is directed to a method for interactive control of selection and/or analysis of regions of interest (ROIs) detected within a medical image of a subject [e.g., a three-dimensional image, e.g., a nuclear medicine image (e.g., bone scan (scintigraphy), PET, and/or SPECT), e.g., an anatomical image (e.g., CT, X-ray, or MRI), e.g., a combined nuclear medicine and anatomical image (e.g., overlaid)] and representing potential lesions, the method comprising: (a) receiving and/or accessing, by a processor of a computing device, (i) an identification of a plurality of ROIs (e.g., hotspots) having been detected within the medical image [e.g., using a machine learning model], and, (ii) a set of ROI feature values comprising, for each particular ROI of the plurality of detected ROIs, corresponding value(s) of at least one ROI feature {e.g., representing a measure of intensity and/or size of the particular ROI; e.g., a lesion score representing an estimated severity of the underlying lesion represented by the particular ROI; e.g., a determination of lesion type of the lesion represented by the particular ROI; e.g., a likelihood value representing e.g., representing a certainty/confidence in detection of the particular ROI; e.g., and/or a likelihood that the particular ROI represents a true physical lesion within the subject [e.g., the corresponding ROI feature value having been determined (e.g., as output) by the machine learning model upon and/or together with detection of the particular ROI] [e.g., a measure of intensity, size, and/or other graphical feature or combination of features of the particular ROI]}; (b) causing, by the processor, display of a graphical control element allowing for user selection of a subset of the plurality of detected ROIs via user adjustment of one or more displayed indicator widgets within the graphical control element from which values of one or more user-selected criteria (e.g., thresholds) are received; (c) determining, by the processor, based on a user adjustment of [e.g., a position (e.g., of a slider along a scale); e.g., a status (e.g., of a checkbox, radio button, etc.); e.g., an entry or selection of alphanumeric character(s) within (e.g., wherein the indicator widget(s) comprises a text box, drop down menu, or the like)] the one or more displayed indicator widgets, user selected values of the one or more criteria (e.g., thresholds); (d) selecting, by the processor, a user-selected subset of the plurality of detected ROIs based on (i) the set of ROI feature values and (ii) the user selected values of the one or more criteria (e.g., thresholds); and (e) storing and/or providing (e.g., rendering), by the processor, for display and/or further processing, an identification of the user-selected subset.

In certain embodiments, a particular one of the one or more criteria is a rank threshold whose value corresponds to a position on an ordered (e.g., ranked) list, and the method comprises: at step (c), determining the value of the rank threshold; and at step (d), ordering the plurality of detected ROIs according to their corresponding feature values in the set of ROI feature values, thereby creating an ordered list of ROIs and selecting, as the user-selected subset, those ROIs having a position in the ordered list of ROIs above and/or below the value of the rank threshold.

In certain embodiments, the at least one ROI feature is or comprises one or more of (i) to (iii) as follows: (i) a ROI size (e.g., volume) that provides a measure of size of a particular ROI, (ii) a ROI intensity (e.g., voxel intensity value) that provides a measure of intensity a particular ROI, and (iii) an intensity-weighted ROI size, providing a measure of (e.g., and determined as a function of) both size and intensity of a particular ROI.

In certain embodiments, the at least one ROI feature is or comprises a lesion classification (e.g., miTNM classification) that classifies a given ROI according to a particular lesion labeling and classification scheme (e.g., an miTNM classification scheme).

In certain embodiments, the at least one ROI feature is or comprises a lesion location identifying an anatomical location of an underlying physical lesion that a given ROI represents [e.g., wherein the anatomical location is a tissue region or organ selected from a (e.g., pre-defined) set of locations {e.g., wherein at least one member of the set of locations is a skeletal region identifying ROIs representing lesions within bone of the subject; e.g., wherein at least one member of the set of locations is a prostate region identifying ROIs representing lesions with a prostate of the subject; e.g., wherein at least one member of the set of locations is a lymph region, identifying ROIs representing lesions within lymph nodes of the subject; e.g., wherein one or more members of the set of locations is/are one or more particular lymph node regions and/or lymph sub-regions (e.g., pelvic lymph) identifying ROIs representing lesions within particular lymph nodes and/or lymph sub-regions (e.g., pelvic lymph) within the subject; e.g., wherein one or more members of the set of locations is a liver region identifying ROIs representing lesions within a liver of the subject; e.g., wherein one or more members of the set of locations is an arm region identifying ROIs representing lesions within one or both arms (e.g., at least a portion of one or both humerus bones) the subject; e.g., wherein one or more members of the set of locations is a skull region identifying ROIs representing lesions within a skull or head of the subject}].

In certain embodiments, the at least one ROI feature is or comprises a likelihood value [e.g., having been determined (e.g., as output) by the machine learning model upon and/or together with detection of a given ROI] and representing a likelihood (e.g., as determined by the machine learning model) that the given ROI represents a true physical lesion within the subject.

In certain embodiments, the one or more criteria [e.g., specified by the user via adjustment of the one or more displayed indicator widgets, e.g., via user entry of alphanumeric character(s) within a textbox of the displayed widget(s) and/or via user selection of alphanumeric characters presented to the user via a drop-down menu of the displayed widget(s)] comprises one or more of (i)-(iii) as follows: (i) tumor/lesion type classification [e.g., miTNM classification, e.g., local tumors (T), regional nodes (N), and/or distant metastases (M)] [e.g., such that user adjustment of the one or more displayed indicator widgets causes selection of all ROIs having a particular user-specified miTNM classification (e.g., the particular user-specified miTNM classification being the user-selected value of the tumor/lesion type classification criteria)], (ii) a measure of ROI intensity [e.g., such as a standardized uptake value (SUV) (e.g., such that user adjustment of the one or more displayed indicator widgets causes selection of all ROIs exceeding a user-identified minimum threshold SUV value); e.g., where SUV reflects intensity of the ROI in relation to a reference intensity level], and (iii) a measure of ROI volume (e.g., such that user adjustment of the one or more displayed indicator widgets causes selection of all ROIs exceeding a user-identified minimum volume, e.g., a normalized volume).

In certain embodiments, the one or more criteria comprise a ROI likelihood threshold [e.g., such that user adjustment of the one or more displayed indicator widgets causes selection of all ROIs having corresponding (e.g., machine-learning model-determined) likelihood values that are equal to and/or exceed the ROI likelihood value].

In certain embodiments, the method comprises causing, by the processor, graphical rendering of one or both of (i) the plurality of detected ROIs and/or (ii) the user-selected subset of the plurality of detected ROIs, wherein each detected ROI is rendered as a (e.g., colorized) graphical shape and/or outline thereof (e.g., identifying/demarcating the ROI) overlaid on the medical image.

In certain embodiments, the method comprises causing, by the processor, rendering a plurality of graphical shapes as overlaid on the medical image, each of the plurality of graphical shapes corresponding to and demarking a detected ROI and having a solid, partially transparent, fill (e.g., colored fill); receiving, by the processor, via a user interaction with an opacity setting graphical widget, an opacity value (e.g., ranging from totally opaque to totally transparent); and updating an opacity of the solid fill and/or boundary of the graphical shapes according to the user-selected opacity value.

In certain embodiments, the method comprises: causing, the processor, rendering, for each detected ROI, a graphical outline demarcating a boundary of the ROI overlaid on the medical image [e.g., the graphical outline is unfilled, such that voxels of the medical image within the outline are unobscured; e.g., wherein the graphical outline is rendered to (e.g., directly) follow boundaries of individual voxels of the medical image; e.g., wherein the rendered outline is smoothed] {e.g., wherein the method comprises receiving a user-selected outline style (e.g., filled area with outline, filled area only, outline only, outline follows boundaries of individual pixels of the medical image, outline follows a smoothed curve of individual pixels of the medical image) and causing rendering of the detected ROIs according to the user-selected outline style}.

In certain embodiments, the method comprises receiving, by the processor, a user selection of a particular hanging protocol (e.g., simultaneous display of axial, coronial, and sagittal projections of the medical image) and causing, by the processor, display of the medical image according to the particular (i.e., user-selected) hanging protocol (e.g., within an image viewing and/or analysis graphical user interface (GUI)).

In certain embodiments, the set of ROI feature values comprises, for each particular ROI of the plurality of detected ROIs, a lesion location assignment having an initial value: (i) identifying a particular anatomical region in which the particular ROI (e.g., the potential physical lesion that it represents) is (e.g., determined, by the processor to be) located or (ii) identifying the particular ROI has as unassigned [e.g., having an indeterminate or as yet-to-be specified location; e.g., not associated with any particular anatomical region (e.g., to be determined)]; the one or more user-selected criteria comprise a lesion location assignment criteria; and the method comprises: at step (c), determining, via the user interaction with the one or more displayed indicator widgets, as the value of the lesion location assignment criteria, an unassigned ROIs value (e.g., thereby causing selection of all unassigned ROIs); at step (d), selecting, by the as the user-selected subset, all unassigned ROIs; causing, by the processor, graphical rendering and display of the user-selected subset; and for each of particular ROI of at least a portion of the user-selected subset (e.g., the unassigned ROIs): receiving, by the processor, a user input (e.g., selection) of a location assignment for the particular ROI; and updating the lesion location assignment for the particular ROI with the user input (e.g., selected) location assignment.

In certain embodiments, the method comprises receiving, by the processor, a user selection of a particular ROI of the plurality of detected ROIs [e.g., optionally, causing rendering of the user selected ROI in a manner that visually emphasizes and/or differentiates the user-selected ROI relative to other (e.g., un-selected) ROIs (e.g., hiding/obscuring/otherwise differentiating from the un-selected ROIs)]; receiving, by the processor a user selection of one or more voxels of the medical image to add to, and/or subtract from the particular (i.e., user-selected) ROI; and updating the particular (i.e., user-selected) ROI to incorporate and/or exclude the one or more user-selected voxels.

In certain embodiments, at least a portion of the medical image is rendered and display for user viewing and/or review within a graphical user interface (GUI) and the method comprises: receiving, by the processor, one or more user-identified points (e.g., voxels) within the medical image, each of the one or more user-identified points corresponding to a location of a user single-click within the GUI; for each particular one of the one or more user-identified points within the medical image, segmenting, by the processor, the medical image to delineate a 3D volume of corresponding user-specified ROI using (i) the particular user-identified point and (ii) intensities of voxels of the medical image about [e.g., in proximity to; e.g., within a sub-volume of the medical image enclosing (e.g., centered on) the particular user-identified point (e.g., the sub-volume defined by one or more threshold distances (e.g., a spherical volume centered on and comprising voxels within a threshold distance/radius from the particular user-identified point; e.g., a rectangular volume with particular x,y,z dimensions centered on the user-identified point)] the particular user-identified point, thereby determining one or more user-specified ROIs, each associated with and segmented via a user single-click; and updating, by the processor, the initial set of ROIs to include the plurality of user-specified ROIs.

In certain embodiments, for each particular one of the one or more user-identified points within the medical image, segmenting the medical image to delineate the 3D volume of the corresponding user-specified ROIs comprises: determining a local intensity threshold value based on the intensities of the voxels of the medical image about the particular user-identified point [e.g., and one or more reference intensity values, each associated with and determined using intensities of voxels within a corresponding reference tissue region (e.g., a liver; e.g., an aorta portion; e.g., parotid)]; and using the local intensity threshold value to segment the medical image to delineate the 3D volume of the corresponding user-specified ROI.

In certain embodiments, for each particular one of the one or more user-identified points within the medical image, segmenting the medical image to delineate the 3D volume of the corresponding user-specified ROI comprises: determining an initial intensity threshold value using the particular user-identified point; at a first step, using the initial intensity threshold value to segment the medical image to identify and delineate a connected region within the medical image and determining an updated intensity threshold value based on intensities of the medical image within the connected region; and at a second step (e.g., iteration), segmenting the medical image to identify and delineate an updated connected region updating the intensity threshold value based on intensities of the medical image within the updated connected region.

In certain embodiments, the medical image is or comprises a 3D functional image. In certain embodiments, the medical image is or comprises a nuclear medicine image. In certain embodiments, the medical image is or comprises a positron emission tomography (PET) image and/or a single photon emission computed tomography (SPECT) image obtained following administration of an agent to the subject. In certain embodiments, the agent comprises a PSMA binding agent. In certain embodiments, the medical image is or comprises a PET image. In certain embodiments, the agent comprises ¹⁸F. In certain embodiments, the agent is or comprises [18F]DCFPyL. In certain embodiments, the agent is or comprises PSMA-11. In certain embodiments, the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

In another aspect, the invention is directed to a method for allowing a user to interactively segment a 3D medical image to detect and delineate 3D volumes of hotspots representing potential cancerous lesions within a subject via a single-click, the method comprising: (a) receiving and/or accessing, by a processor of a computing device, the 3D medical image; (b) causing, by the processor, rendering and display of the 3D medical image within an interactive image viewer and/or analysis graphical user interface (GUI); (c) receiving, by the processor, one or more user-identified points (e.g., voxels) within the 3D medical image, each of the one or more user-identified points corresponding to a location of a single-click, by the user, within the GUI; (d) for each particular one of the one or more user-identified points within the medical image, segmenting, by the processor, the medical image to delineate a 3D volume of corresponding user-specified hotspot using (i) the particular user-identified point and (ii) intensities of voxels of the medical image about [e.g., in proximity to; e.g., within a sub-volume of the medical image enclosing (e.g., centered on) the particular user-identified point (e.g., the sub-volume defined by one or more threshold distances (e.g., a spherical volume centered on and comprising voxels within a threshold distance/radius from the particular user-identified point; e.g., a rectangular volume with particular x,y,z dimensions centered on the user-identified point)] the particular user-identified point, thereby creating a set of user-specified hotspots, each associated with and segmented via a single-click; and (e) storing and/or providing, by the processor, for display and/or further processing, the set of user-specified hotspots.

In certain embodiments, for each particular one of the one or more user-identified points within the medical image, segmenting the medical image to delineate the 3D volume of the corresponding user-specified hotspot comprises: determining a local intensity threshold value based on the intensities of the voxels of the medical image about the particular user-identified point [e.g., and one or more reference intensity values, each associated with and determined using intensities of voxels within a corresponding reference tissue region (e.g., a liver; e.g., an aorta portion; e.g., parotid)]; and using the local intensity threshold value to segment the medical image to delineate the 3D volume of the corresponding user-specified hotspot.

In certain embodiments, for each particular one of the one or more user-identified points within the medical image, segmenting the medical image to delineate the 3D volume of the corresponding user-specified hotspot comprises: determining an initial intensity threshold value using the particular user-identified point; at a first step, using the initial intensity threshold value to segment the medical image to identify and delineate a connected region within the medical image and determining an updated intensity threshold value based on intensities of the medical image within the connected region; and at a second step (e.g., iteration), segmenting the medical image to identify and delineate an updated connected region updating the intensity threshold value based on intensities of the medical image within the updated connected region.

In certain embodiments, the medical image is or comprises a 3D functional image. In certain embodiments, the medical image is or comprises a nuclear medicine image. In certain embodiments, the medical image is or comprises a positron emission tomography (PET) image and/or a single photon emission computed tomography (SPECT) image obtained following administration of an agent to the subject. In certain embodiments, the agent comprises a PSMA binding agent. In certain embodiments, the medical image is or comprises a PET image. In certain embodiments, the agent comprises ¹⁸F. In certain embodiments, the agent is or comprises [18F]DCFPyL. In certain embodiments, the agent is or comprises PSMA-11. In certain embodiments, the agent comprises one or more members selected from the group consisting of ^{99m}Tc, ⁶⁸Ga, ¹⁷⁷Lu, ²²⁵Ac, ¹¹¹In, ¹²³I, ¹²⁴j, and ¹³¹I.

In another aspect, the invention is directed to a method for detecting hotspots (e.g., regions of cancer and/or regions suspected of being cancer, e.g., metastases) in a high uptake organ (e.g., liver), the method comprising: (a) receiving and/or accessing, by a processor of a computing device, a 3D functional image [e.g., a nuclear medicine image (PET, and/or SPECT)] of a subject; (b) receiving and/or accessing, by the processor, a segmentation mask (e.g., aligned with the functional image, e.g., identifying one or more particular organs and/or bones) identifying a region of the 3D functional image corresponding to high-uptake organ; (c) determining, by the processor, a local background intensity value associated with and representing background uptake within the high uptake organ using the 3D functional image and the segmentation mask; (d) determining [e.g., identifying and delineating (e.g., segmenting)], by the processor, within the 3D functional image, one or more 3D hotspot volumes, wherein each of the one or more 3D hotspot volumes represents a potential cancerous lesion within the high-uptake organ; and (e) storing and/or providing (e.g., rendering), by the processor, for display and/or further processing, the one or more 3D hotspot volumes.

In certain embodiments, step (c) comprises: using the segmentation mask to identify a volume of interest (VOI) within the 3D functional image corresponding to the high uptake organ (e.g., overlaying/transferring/mapping the segmentation mask onto the 3D functional image); fitting a multi-component mixture model to intensities of voxels of the 3D functional image within the VOI; and determining the local background intensity value based at least in part on the multi-component mixture model having been fit to the intensities of the voxels within the VOI (e.g., as a mean of a major mode of the multi-component mixture model; e.g., based on a mean and/or a standard deviation of a major mode of the multi-component mixture model).

In certain embodiments, step (d) comprises: determining a detection threshold value based on the local background intensity value; identifying, within the 3D functional image (e.g., within a volume of interest of the 3D functional image corresponding to the high-uptake organ), one or more (e.g., preliminary) sub-regions representing prospective hotspots, using the segmentation threshold value; and determining (e.g., segmenting) the one or more 3D hotspot volumes based on the one or more identified sub-regions.

In certain embodiments, step (d) comprises: identifying, within the 3D functional image (e.g., within a volume of interest of the 3D functional image corresponding to the high-uptake organ), one or more preliminary sub-regions representing prospective hotspots; determining, for each of the one or more preliminary sub-regions, corresponding values for one or more selection criteria; selecting at least a portion of the one or more preliminary sub-regions based on the corresponding selection criteria values; and determining the one or more 3D hotspot volumes based on the selected portion of the one or more preliminary sub-regions.

In certain embodiments, the one or more selection criteria comprise(s) a degree of overlap (e.g., absolute volume, volumetric percentage) between a given one of the one or more preliminary sub-regions and a region (e.g., 3D volume of interest) of the 3D functional image corresponding to the high-uptake organ [e.g., wherein/such that step (d) comprises, determining, for each particular one of the one or more preliminary sub-regions, a corresponding value measuring its degree of overlap and the region of the 3D functional image corresponding to the high-uptake organ].

In certain embodiments, the one or more selection criteria comprise(s) a degree of overlap (e.g., absolute volume, volumetric percentage) between a given one of the one or more preliminary sub-regions and a region (e.g., a 3D volume) of the 3D functional image corresponding to at least one other organ or tissue region [e.g., a neighboring organ, an organ with high physiological uptake, an organ known to “bleed over” (e.g., distort uptake values of a neighboring organ due to a misalignment between a functional image and a segmentation mask) into the high uptake organ, e.g., kidney] [e.g., wherein/such that step (d) comprises, determining, for each particular one of the one or more preliminary sub-regions, a corresponding value measuring its degree of overlap and the region of the 3D functional image corresponding to the at least one other organ or tissue region].

In certain embodiments, the one or more selection criteria comprise(s) a minimum (e.g., hotspot) volume (e.g., a minimum number of voxels; e.g., a minimum corresponding physical volume) [e.g., such that only preliminary sub-regions having volumes greater than or equal to the minimum (e.g., hotspot) volume are selected] [e.g., wherein the minimum hotspot volume is at least 2 (e.g., 1, 5, 10) voxels].

In certain embodiments, step (d) comprises smoothing at least a portion of the 3D functional image (e.g., a VOI within the 3D functional image corresponding to the high-uptake organ).

In certain embodiments, step (d) comprises: identifying, within the 3D functional image (e.g., within a volume of interest of the 3D functional image corresponding to the high-uptake organ), one or more preliminary sub-regions representing prospective hotspots; and for each particular preliminary sub-region of at least a portion of the one or more preliminary sub-regions, segmenting at least a portion of the 3D functional image (e.g., a region of the 3D functional image overlapping and/or in proximity to the particular preliminary sub-region) to determine (e.g., delineate) a corresponding 3D hotspot volume, thereby determining the one or more 3D hotspot volumes {e.g., performing one or more of steps/sub-steps (i) through (v), as follows: (i) identifying a VOI within the 3D functional image corresponding to the high-uptake organ; (ii) smoothing intensities of voxels within the identified VOI, thereby obtaining a set of smoothed intensities; (iii) sub-dividing the identified VOI into one or more sub-segments [e.g., using a watershed algorithm (e.g., with peaks of the preliminary 3D hotspot volumes as basins); e.g., based on the set of smoothed intensities] obtain the smoothed part of the 3D functional image; (iv) identifying, for each particular one of the one or more preliminary sub-regions, a corresponding sub-segment of the VOI with which the particular preliminary sub-region overlaps; and (v) for each particular one of the one or more preliminary sub-regions, segmenting the corresponding 3D hotspot volume based on one or more of (A) through (C) as follows: (A) a measure of (e.g., maximal) intensity (e.g., a SUVmax) of the particular preliminary sub-region, (B) a detection threshold determined based on the local background intensity value, and (C) the corresponding sub-segment of the VOI with which the particular preliminary sub-region overlaps.}

In certain embodiments, the high-uptake organ is or comprises a liver of the subject.

In another aspect, the invention is directed to a system for interactive control of selection and/or analysis of hotspots (e.g., regions of cancer and/or regions suspected of being cancer, e.g., metastases) detected within a medical image [e.g., a three-dimensional image, e.g., a nuclear medicine image (e.g., bone scan (scintigraphy), PET, and/or SPECT), e.g., an anatomical image {e.g., computed tomography (CT), X-ray, or magnetic resonance imaging (MRI) scan}, e.g., a combined nuclear medicine and anatomical image (e.g., overlaid)] of a subject and representing potential lesions, the system comprising: a processor of a computing device; and memory having instructions stored thereon, wherein the instructions, when executed by the processor, cause the processor to: (a) receive and/or access (i) an identification of a plurality of hotspots having been detected within the medical image using a machine learning model, and, (ii) a set of hotspot feature values comprising, for each particular hotspot of the plurality of detected hotspots, a corresponding value of at least one hotspot feature representing and/or indicative of a certainty or confidence in detection of the particular hotspot (e.g., a measure/determination of intensity, size, lesion type, and/or other graphical feature or combination of features of the particular hotspot) and/or a likelihood that the particular hotspot represents a true physical lesion within the subject [e.g., a corresponding likelihood value having been determined (e.g., as output) by the machine learning model upon and/or together with detection of the particular hotspot]; (b) cause display of a graphical control element allowing for user selection of a subset of the plurality of detected hotspots via user adjustment of one or more displayed indicator widgets within the graphical control element from which values of one or more criteria (e.g., thresholds) are determined; (c) determine, based on a user adjustment of [e.g., a position (e.g., of a slider along a scale) of; e.g., a status (e.g., of a checkbox, radio button, etc.) of; e.g., an entry or selection of alphanumeric character(s) within (e.g., wherein the indicator widget comprises a text box, drop down menu, or the like)] the one or more displayed indicator widgets, user selected values of the one or more criteria (e.g., thresholds); (d) select a user-selected subset of the plurality of initial hotspots based on (i) the set of hotspot feature values and (ii) the user selected values of the one or more criteria (e.g., thresholds); and (e) store and/or provide (e.g., render), for display and/or further processing, an identification of the user-selected subset.

In certain embodiments, the system comprises one or more features described herein, for example in paragraphs above [e.g., with reference to methods for interactive control of selection and/or analysis of hotspots detected within a medical image of a subject and representing potential lesions (e.g., at paragraphs [0015]-[0031])].

In another aspect, the invention is directed to a system for interactive control of selection and/or analysis of regions of interest (ROIs) detected within a medical image of a subject [e.g., a three-dimensional image, e.g., a nuclear medicine image (e.g., bone scan (scintigraphy), PET, and/or SPECT), e.g., an anatomical image (e.g., CT, X-ray, or MRI), e.g., a combined nuclear medicine and anatomical image (e.g., overlaid)] and representing potential lesions, the system comprising: a processor of a computing device; and memory having instructions stored thereon, wherein the instructions, when executed by the processor, cause the processor to: (a) receive and/or access (i) an identification of a plurality of ROIs (e.g., hotspots) having been detected within the medical image [e.g., using a machine learning model], and, (ii) a set of ROI feature values comprising, for each particular ROI of plurality of detected ROIs, corresponding value(s) of at least one ROI feature {e.g., representing a measure of intensity and/or size of the particular ROI; e.g., a lesion score representing an estimated severity of the underlying lesion represented by the particular ROI; e.g., a determination of lesion type of the lesion represented by the particular ROI; e.g., a likelihood value representing e.g., representing a certainty/confidence in detection of the particular ROI; e.g., and/or a likelihood that the particular ROI represents a true physical lesion within the subject [e.g., the corresponding ROI feature value having been determined (e.g., as output) by the machine learning model upon and/or together with detection of the particular ROI] [e.g., a measure of intensity, size, and/or other graphical feature or combination of features of the particular ROI]}; (b) cause display of a graphical control element allowing for user selection of a subset of the plurality of detected ROIs via user adjustment of one or more displayed indicator widgets within the graphical control element from which values of one or more user-selected criteria (e.g., thresholds) are received; (c) determine, based on a user adjustment of [e.g., a position (e.g., of a slider along a scale); e.g., a status (e.g., of a checkbox, radio button, etc.); e.g., an entry or selection of alphanumeric character(s) within (e.g., wherein the indicator widget(s) comprises a text box, drop down menu, or the like)] the one or more displayed indicator widgets, user selected values of the one or more criteria (e.g., thresholds); (d) select a user-selected subset of the plurality of detected ROIs based on (i) the set of ROI feature values and (ii) the user selected values of the one or more criteria (e.g., thresholds); and (e) store and/or provide (e.g., render) for display and/or further processing, an identification of the user-selected subset.

In certain embodiments, the system comprises one or more features described herein, for example in paragraphs above [e.g., with reference to a method for interactive control of selection and/or analysis of regions of interest (ROIs) detected within a medical image of a subject and representing potential lesions (e.g., at paragraphs [0033]-[0049])].

In another aspect, the invention is directed to a system for allowing a user to interactively segment a 3D medical image to detect and delineate 3D volumes of hotspots representing potential cancerous lesions within a subject via a single-click, the system comprising: a processor of a computing device; and memory having instructions stored thereon, wherein the instructions, when executed by the processor, cause the processor to: (a) receive and/or access the 3D medical image; (b) cause render and display of the 3D medical image within an interactive image viewer and/or analysis graphical user interface (GUI); (c) receive one or more user-identified points (e.g., voxels) within the 3D medical image, each of the one or more user-identified points corresponding to a location of a single-click, by the user, within the GUI; (d) for each particular one of the one or more user-identified points within the medical image, segment the medical image to delineate a 3D volume of corresponding user-specified hotspot using (i) the particular user-identified point and (ii) intensities of voxels of the medical image about [e.g., in proximity to; e.g., within a sub-volume of the medical image enclosing (e.g., centered on) the particular user-identified point (e.g., the sub-volume defined by one or more threshold distances (e.g., a spherical volume centered on and comprising voxels within a threshold distance/radius from the particular user-identified point; e.g., a rectangular volume with particular x,y,z dimensions centered on the user-identified point)] the particular user-identified point, thereby creating a set of user-specified hotspots, each associated with and segmented via a single-click; and (e) store and/or provide (e.g., render) for display and/or further processing, the set of user-specified hotspots.

In certain embodiments, the system comprises one or more features described herein, for example in paragraphs above [e.g., with reference to a method for allowing a user to interactively segment a 3D medical image to detect and delineate 3D volumes of hotspots representing potential cancerous lesions within a subject via a single-click (e.g., at paragraphs [0051]-[0053])].

In another aspect, the invention is directed to a system for detecting hotspots (e.g., regions of cancer and/or regions suspected of being cancer, e.g., metastases) in a high uptake organ (e.g., liver), the system comprising: a processor of a computing device; and memory having instructions stored thereon, wherein the instructions, when executed by the processor, cause the processor to: (a) receive and/or access a 3D functional image [e.g., a nuclear medicine image (PET, and/or SPECT)] of a subject; (b) receive and/or access a segmentation mask (e.g., aligned with the functional image, e.g., identifying one or more particular organs and/or bones) identifying a region of the 3D functional image corresponding to high-uptake organ; (c) determine a local background intensity value associated with and representing background uptake within the high uptake organ using the 3D functional image and the segmentation mask; (d) determine [e.g., identify and delineate (e.g., segment)] within the 3D functional image, one or more 3D hotspot volumes, wherein each of the one or more 3D hotspot volumes represents a potential cancerous lesion within the high-uptake organ; and (e) store and/or provide (e.g., render) for display and/or further processing, the one or more 3D hotspot volumes.

In certain embodiments, the system comprises one or more features described herein, for example in paragraphs above [e.g., with reference to a method for detecting hotspots in a high uptake organ (e.g., at paragraphs [0055]-[0063])].

In certain embodiments, elements described with respect to one aspect of the invention are implemented in another aspect of the invention described herein.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1A is a set of three images showing corresponding slices of a CT image, a PET image, and a PET/CT fusion, obtained from a 3D PET/CT scan, according to an illustrative embodiment.

FIG. 1B is an image showing a set of two slices of a PET/CT composite image in which a PET image is overlaid on a CT scan, according to an illustrative embodiment.

FIG. 2 is a diagram illustrating an example process for segmenting an anatomical image and identifying anatomical boundaries in a co-aligned functional image, according to an illustrative embodiment.

FIG. 3 is a diagram illustrating an example process for segmenting and classifying hotspots, according to an illustrative embodiment.

FIG. 4A is a screenshot of a graphical user interface showing segmented anatomical regions corresponding to arms and a skull of a subject, according to an illustrative embodiment.

FIG. 4B is a block flow diagram of an example process for detecting hotspots in a high uptake organ, according to an illustrative embodiment.

FIG. 5 is a schematic showing an approach for computing lesion index values, according to an illustrative embodiment.

FIG. 6A is a block flow diagram of an example process for interactive control of selection and/or analysis of regions of interest (ROIs) detected within a medical image, according to an illustrative embodiment.

FIG. 6B is a screenshot of a graphical user interface for display and analysis of nuclear medicine images and/or hotspots detected therein, according to an illustrative embodiment.

FIG. 7A is a constructive mock-up of a graphical user interface including a graphical lesion selection widget, according to an illustrative embodiment.

FIG. 7B is a constructive mock-up of a graphical user interface including a graphical lesion selection widget, according to an illustrative embodiment.

FIG. 7C depicts a graphical control element (graphical user interface GUI widget) including a text box and confirmation button, according to an illustrative embodiment.

FIG. 8 is a screenshot of a GUI including a graphical hotspot transparency selection widget, according to an illustrative embodiment.

FIG. 9 is a screenshot of a GUI comprising a graphical rendering of a medical image with outlines of detected hotspots overlaid, according to an illustrative embodiment.

FIG. 10 is a screenshot of a GUI including a simultaneous presentation of axial, coronal, and sagittal image projections, according to an illustrative embodiment.

FIG. 11 is a screenshot of a graphical control element for filtering and selecting hotspots, according to an illustrative embodiment.

FIG. 12 is a screenshot of a graphical control element for setting hotspot selection thresholds, according to an illustrative embodiment.

FIG. 13A is a screenshot of a graphical user interface including a graphical editing of hotspot segmentation widget, according to an illustrative embodiment.

FIG. 13B is a block flow diagram of an example process for user assisted, semi-automated, hotspot segmentation via a single-click, according to an illustrative embodiment.

FIG. 13C is a screenshot showing transversal and coronal views of a medical image and a user-identified point and sub-region thereof, according to an illustrative embodiment.

FIG. 13D is a screenshot showing transversal and coronal views of a medical image and a segmented hotspot, according to an illustrative embodiment.

FIG. 14 is a block diagram of an exemplary cloud computing environment, used in certain embodiments.

FIG. 15 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Certain Definitions

In order for the present disclosure to be more readily understood, certain terms are first defined below. Additional definitions for the following terms and other terms are set forth throughout the specification.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

About, approximately: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

First, second, etc.: It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner. In addition, unless stated otherwise, a set of elements may comprise one or more elements.

Image: As used herein, an “image”—for example, a three-dimensional (3D) image of subject, includes any visual representation, such as a photo, a video frame, streaming video, as well as any electronic, digital or mathematical analogue of a photo (e.g., a digital image), video frame, or streaming video, displayed or stored in memory (e.g., a digital image may, but need not be displayed for visual inspection). Any apparatus described herein, in certain embodiments, includes a display for displaying an image or any other result produced by the processor. Any method described herein, in certain embodiments, includes a step of displaying an image or any other result produced via the method. In certain embodiments, an image is a 3D image, conveying information that varies with position within a 3D volume. Such images may, for example, be represented digitally as a 3D matrix (e.g., a N×M×L matrix) with each voxel of a 3D image represented by an element of a 3D matrix. Other representations are also contemplated and included, for example, a 3D matrix may be reshaped as a vector (e.g., a 1×K size vector, where K is a total number of voxels) by stitching each row or column end to end. Examples of images include, for example, medical images, such as bone-scan images (also referred to as scintigraphy images), computed tomography (CT) images, magnetic resonance images (MRIs), optical images (e.g., bright-field microscopy images, fluorescence images, reflection or transmission images, etc.), positron emission tomography (PET) images, single-photon emission tomography (SPECT) images, ultrasound images, x-ray images, and the like. In certain embodiments, a medical image is or comprises a nuclear medicine image, produced from radiation emitted from within a subject being imaged. In certain embodiments, a medical image is or comprises an anatomical image (e.g., a 3D anatomical image) conveying information regarding location and extent of anatomical structures such as internal organs, bones, soft-tissue, and blood vessels, within a subject. Examples of anatomical images include, without limitation, x-ray images, CT images, MRIs, and ultrasound images. In certain embodiments, a medical image is or comprises a functional image (e.g., a 3D functional image) conveying information relating to physiological activities within specific organs and/or tissue, such as metabolism, blood flow, regional chemical composition, absorption, etc. Examples of functional images include, without limitation, nuclear medicine images, such as PET images, SPECT images, as well as other functional imaging modalities, such as functional MRI (fMRI), which measures small changes in blood flow for use in assessing brain activity.

Map: As used herein, the term “map” is understood to mean a visual display, or any data representation that may be interpreted for visual display, which contains spatially-correlated information. For example, a three-dimensional map of a given volume may include a dataset of values of a given quantity that varies in three spatial dimensions throughout the volume. A three-dimensional map may be displayed in two-dimensions (e.g., on a two-dimensional screen, or on a two-dimensional printout).

Segmentation map: As used herein, the term “segmentation map” refers to a computer representation that identifies one or more 2D or 3D regions determined by segmenting an image. In certain embodiments, a segmentation map distinguishably identifies multiple different (e.g., segmented) regions, allowing them to be individually and distinguishably accessed and operated upon and/or used for operating on, for example, one or more images.

3D, three-dimensional: As used herein, “3D” or “three-dimensional” with reference to an “image” means conveying information about three dimensions. A 3D image may be rendered as a dataset in three dimensions and/or may be displayed as a set of two-dimensional representations, or as a three-dimensional representation. In certain embodiments, a 3D image is represented as voxel (e.g., volumetric pixel) data.

Whole body: As used herein, the terms “full body” and “whole body” used (interchangeably) in the context of segmentation and other manners of identification of regions within an image of a subject refer to approaches that evaluate a majority (e.g., greater than 50%) of a graphical representation of a subject's body in a 3D anatomical image to identify target tissue regions of interest. In certain embodiments, full body and whole-body segmentation refers to identification of target tissue regions within at least an entire torso of a subject. In certain embodiments, portions of limbs are also included, along with a head of the subject.

Radionuclide: As used herein, “radionuclide” refers to a moiety comprising a radioactive isotope of at least one element. Exemplary suitable radionuclides include but are not limited to those described herein. In some embodiments, a radionuclide is one used in positron emission tomography (PET). In some embodiments, a radionuclide is one used in single-photon emission computed tomography (SPECT). In some embodiments, a non-limiting list of radionuclides includes ^{99m}Tc, ¹¹¹In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹²⁶I, ¹³¹I, ¹¹C, ¹³N, ¹⁵O, ¹⁸F ¹⁵³Sm ¹⁶⁶Ho ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, ⁷⁵Br, ⁷⁶Br, ⁷⁷Br, ⁸⁰Br, ⁸⁰mBr, ⁸²Br, ⁸³Br, ²¹¹At and ¹⁹²Ir.

Radiopharmaceutical: As used herein, the term “radiopharmaceutical” refers to a compound comprising a radionuclide. In certain embodiments, radiopharmaceuticals are used for diagnostic and/or therapeutic purposes. In certain embodiments, radiopharmaceuticals include small molecules that are labeled with one or more radionuclide(s), antibodies that are labeled with one or more radionuclide(s), and antigen-binding portions of antibodies that are labeled with one or more radionuclide(s).

Machine learning module: Certain embodiments described herein make use of (e.g., include) software instructions that include one or more machine learning module(s), also referred to herein as artificial intelligence software. As used herein, the term “machine learning module” refers to a computer implemented process (e.g., function) that implements one or more specific machine learning algorithms in order to determine, for a given input (such as an image (e.g., a 2D image; e.g., a 3D image), dataset, and the like) one or more output values. For example, a machine learning module may receive as input a 3D image of a subject (e.g., a CT image; e.g., an MRI), and for each voxel of the image, determine a value that represents a likelihood that the voxel lies within a region of the 3D image that corresponds to a representation of a particular organ or tissue of the subject. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a CNN module may be carried out via specialized hardware (e.g., via an application specific integrated circuit (ASIC)).

Single-click: As used herein, the phrase/term “single-click” refers to a single, isolated user interaction with, and/or provision of input to, an input device or mechanism for interfacing and providing input to a computer or other computing device. For example, a user may interact with and provide input to a computer workstation via a keyboard and pointing device, such as a mouse, trackball, trackpad, and the like. Accordingly, in certain embodiments, a “single-click” refers a single user click of a mouse button or button of a trackball, trackpad, etc. In certain embodiments, a single-click may refer to single user inputs via input devices or mechanism that do not necessary receive input via actual clicks of a button, but, rather, may use other, analogous, approaches, such as taps of a touchscreen device, user hand gestures recorded via motion capture mechanisms (e.g., as provided and/or contemplated for interaction with augmented or virtual reality computing systems), and the like. Other forms of input (e.g., not limited to actions or gestures with a user's hand(s)) can be received from a user as well, including, without limitation, acoustic, speech, or various other tactile input(s). In such contexts, it should be understood that single-click, analogously, as explained and used herein, refers to a single, minimal, user provision of input.

Subject: As used herein, a “subject” means a human or other mammal (e.g., rodent (mouse, rat, hamster), pig, cat, dog, horse, primate, rabbit, and the like). The term “subject” is used herein interchangeably with the term “patient”.

Administering: As used herein, “administering” an agent means introducing a substance (e.g., an imaging agent) into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments.

Tissue: As used herein, the term “tissue” refers to bone (osseous tissue) as well as soft tissue.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

As described in further detail herein, and, for example, in PCT/EP2021/068337, filed on Jul. 2, 2021, entitled, “SYSTEMS AND METHODS FOR ARTIFICIAL INTELLIGENCE-BASED IMAGE ANALYSIS FOR DETECTION AND CHARACTERIZATION OF LESIONS,” and published on Jan. 13, 2022, as International (PCT) Publication No. WO/2022/008374; and (ii) U.S. application Ser. No. 18/207,246, filed Jul. 8, 2023, entitled, “SYSTEMS AND METHODS FOR ASSESSING DISEASE BURDEN AND PROGRESSION,” the content of each of which are incorporated by reference herein in their entirety, in certain embodiments, automated (including semi-automated) analysis of three dimensional (3D) medical images can be used to detect or evaluate cancer or risk thereof in a patient, and/or to monitor disease progression and treatment efficacy. These 3D medical images may include, for example, positron emission tomography (PET) images, single-photon emission computerized tomography (SPECT) images, whole-body bone images, combined PET/CT images (CT=computed tomography), and/or combined SPECT/CT images. In certain embodiments, a PET image of a patient is obtained using the prostate-specific membrane antigen (PSMA) binding agent PyL™ (also referred to as 18F-DCFPyL, [18F]DCFPyL, or DCFPyL-18F, with chemical structure as shown in the above-referenced WO/2022/008374) and is overlaid (or otherwise used in combination) with a CT image of the patient. Examples of the automated analysis of these composite 18F-DCFPyL PET/CT images are described in the above-referenced WO/2022/008374 and U.S. application Ser. No. 18/207,246.

Analysis of medical images may be used to identify and delineate various regions of interest (ROIs) that represent potential underlying physical lesions within a subject. These ROIs may, for example, be localized regions of elevated intensity (e.g., bright spots)—referred to as hotspots, which appear in nuclear medicine images due to accumulation of imaging agents as described herein—or may be associated with other characteristic image features, e.g., depending on the particular imaging modality and collection protocol. Identified ROIs may be analyzed to determine overall patient risk and survival/prognostic metrics, indicative of disease state, progression, and/or treatment efficacy.

Further to the above, presented herein are systems and methods for facilitating user interaction with—for example for review and/or analysis of—medical images and ROIs identified therein. As described in further detail herein, in particular, the present disclosure provides technologies that allow for efficient and user-friendly interaction with GUIs for viewing and analyzing medical images to facilitate accurate and consistent expert review and input in identification of cancerous lesions within a subject.

### A. Nuclear Medicine Images

Nuclear medicine images may be obtained using a nuclear medicine imaging modality such as bone scan imaging (also referred to as scintigraphy), Positron Emission Tomography (PET) imaging, and Single-Photon Emission Tomography (SPECT) imaging.

In certain embodiments, nuclear medicine images are obtained using imaging agents comprising radiopharmaceuticals. Nuclear medicine images may be obtained following administration of a radiopharmaceutical to a patient (e.g., a human subject), and provide information regarding the distribution of the radiopharmaceutical within the patient.

Nuclear medicine imaging techniques detect radiation emitted from the radionuclides of radiopharmaceuticals to form an image. The distribution of a particular radiopharmaceutical within a patient may be influenced and/or dictated by biological mechanisms such as blood flow or perfusion, as well as by specific enzymatic or receptor binding interactions. Different radiopharmaceuticals may be designed to take advantage of different biological mechanisms and/or particular specific enzymatic or receptor binding interactions and thus, when administered to a patient, selectively concentrate within particular types of tissue and/or regions within the patient. Greater amounts of radiation are emitted from regions within the patient that have higher concentrations of radiopharmaceutical than other regions, such that these regions appear brighter in nuclear medicine images. Accordingly, intensity variations within a nuclear medicine image can be used to map the distribution of radiopharmaceutical within the patient. This mapped distribution of radiopharmaceutical within the patient can be used to, for example, infer the presence of cancerous tissue within various regions of the patient's body. In certain embodiments, intensities of voxels of a nuclear medicine image, for example a PET image, represent standardized uptake values (SUVs) (e.g., having been calibrated for injected radiopharmaceutical dose and/or patient weight).

For example, upon administration to a patient, technetium 99m methylenediphosphonate (^{99m}Tc MDP) selectively accumulates within the skeletal region of the patient, in particular at sites with abnormal osteogenesis associated with malignant bone lesions. The selective concentration of radiopharmaceutical at these sites produces identifiable hotspots—localized regions of high intensity—in nuclear medicine images. Accordingly, presence of malignant bone lesions associated with metastatic prostate cancer can be inferred by identifying such hotspots within a whole-body scan of the patient. In certain embodiments, analyzing intensity variations in whole-body scans obtained following administration of ^{99m}Tc MDP to a patient, such as by detecting and evaluating features of hotspots, can be used to compute, risk indices that correlate with patient overall survival and other prognostic metrics indicative of disease state, progression, treatment efficacy, and the like. In certain embodiments, other radiopharmaceuticals can also be used in a similar fashion to ^{99m}Tc MDP.

In certain embodiments, the particular radiopharmaceutical used depends on the particular nuclear medicine imaging modality used. For example, 18F sodium fluoride (NaF) also accumulates in bone lesions, similar to ^{99m}Tc MDP, but can be used with PET imaging. In certain embodiments, PET imaging may also utilize a radioactive form of the vitamin choline, which is readily absorbed by prostate cancer cells.

In certain embodiments, radiopharmaceuticals that selectively bind to particular proteins or receptors of interest—particularly those whose expression is increased in cancerous tissue may be used. Such proteins or receptors of interest include, but are not limited to tumor antigens, such as CEA, which is expressed in colorectal carcinomas, Her2/neu, which is expressed in multiple cancers, BRCA 1 and BRCA 2, expressed in breast and ovarian cancers; and TRP-1 and -2, expressed in melanoma.

For example, human prostate-specific membrane antigen (PSMA) is upregulated in prostate cancer, including metastatic disease. PSMA is expressed by virtually all prostate cancers and its expression is further increased in poorly differentiated, metastatic and hormone refractory carcinomas. Accordingly, radiopharmaceuticals that comprise PSMA binding agents (e.g., compounds that a high affinity to PSMA) labelled with one or more radionuclide(s) can be used to obtain nuclear medicine images of a patient from which the presence and/or state of prostate cancer within a variety of regions (e.g., including, but not limited to skeletal regions) of the patient can be assessed. In certain embodiments, nuclear medicine images obtained using PSMA binding agents are used to identify the presence of cancerous tissue within the prostate, when the disease is in a localized state. In certain embodiments, nuclear medicine images obtained using radiopharmaceuticals comprising PSMA binding agents are used to identify the presence of cancerous tissue within a variety of regions that include not only the prostate, but also other organs and tissue regions such as lungs, lymph nodes, and bones, as is relevant when the disease is metastatic.

In particular, upon administration to a patient, radionuclide labelled PSMA binding agents selectively accumulate within cancerous tissue, based on their affinity to PSMA. In a similar manner to that described above with regard to ^{99m}Tc MDP, the selective concentration of radionuclide labelled PSMA binding agents at particular sites within the patient produces detectable hotspots in nuclear medicine images. As PSMA binding agents concentrate within a variety of cancerous tissues and regions of the body expressing PSMA, localized cancer within a prostate of the patient and/or metastatic cancer in various regions of the patient's body can be detected, and evaluated. Various metrics that are indicative of and/or quantify severity (e.g., likely malignancy) of individual lesions, overall disease burden and risk for a patient, and the like, can be computed based on automated analysis of intensity variations in nuclear medicine images obtained following administration of a PSMA binding agent radiopharmaceutical to a patient. These disease burden and/or risk metrics may be used to stage disease and make assessments regarding patient overall survival and other prognostic metrics indicative of disease state, progression, treatment efficacy,

A variety of radionuclide labelled PSMA binding agents may be used as radiopharmaceutical imaging agents for nuclear medicine imaging to detect and evaluate prostate cancer. In certain embodiments, the particular radionuclide labelled PSMA binding agent that is used depends on factors such as the particular imaging modality (e.g., PET; e.g., SPECT) and the particular regions (e.g., organs) of the patient to be imaged. For example, certain radionuclide labelled PSMA binding agents are suited for PET imaging, while others are suited for SPECT imaging. For example, certain radionuclide labelled PSMA binding agents facilitate imaging a prostate of the patient, and are used primarily when the disease is localized, while others facilitate imaging organs and regions throughout the patient's body, and are useful for evaluating metastatic prostate cancer.

Several exemplary PSMA binding agents and radionuclide labelled versions thereof are described in further detail in Section H herein, as well as in U.S. Pat. Nos. 8,778,305, 8,211,401, and 8,962,799, and in U.S. Patent Publication No. US 2021/0032206 A1, the content of each of which are incorporated herein by reference in their entireties.

### B. Image Segmentation in Nuclear Medicine Imaging

Nuclear medicine images are functional images. Functional images convey information relating to physiological activities within specific organs and/or tissue, such as metabolism, blood flow, regional chemical composition, and/or absorption. In certain embodiments, nuclear medicine images are acquired and/or analyzed in combination with anatomical images, such as computed tomography (CT) images. Anatomical images provide information regarding location and extent of anatomical structures such as internal organs, bones, soft-tissue, and blood vessels, within a subject. Examples of anatomical images include, without limitation, x-ray images, CT images, magnetic resonance images, and ultrasound images.

Accordingly, in certain embodiments, anatomical images can be analyzed together with nuclear medicine images in order to provide anatomical context for the functional information that they (nuclear medicine images) convey. For example, while nuclear medicine images, such as PET and SPECT convey a three-dimensional distribution of radiopharmaceutical within a subject, adding anatomical context from an anatomical imaging modality, such as CT imaging, allows one to determine the particular organs, soft-tissue regions, bones, etc. that radiopharmaceutical has accumulated in.

For example, a functional image may be aligned with an anatomical image so that locations within each image that correspond to a same physical location—and therefore correspond to each other—can be identified. For example, coordinates and/or pixels/voxels within a functional image and an anatomical image may be defined with respect to a common coordinate system, or a mapping (i.e., a functional relationship) between voxels within the anatomical image and voxels within the functional image established. In this manner, one or more voxels within an anatomical image and one or more voxels within a functional image that represent a same physical location or volume can be identified as corresponding to each other.

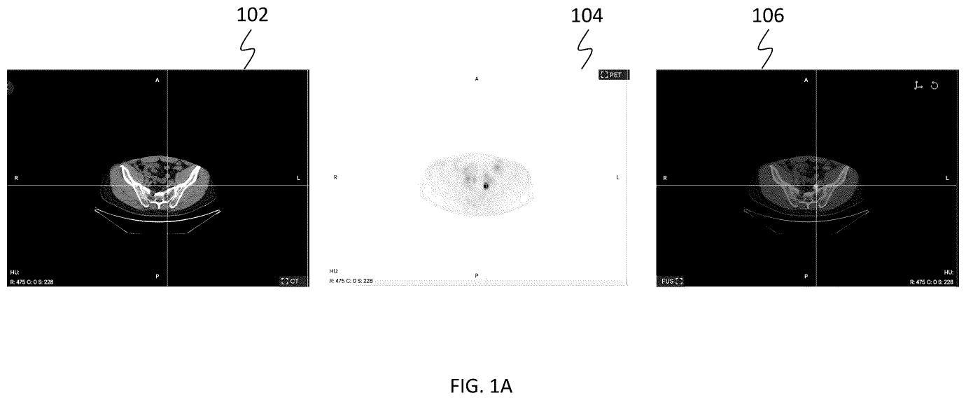

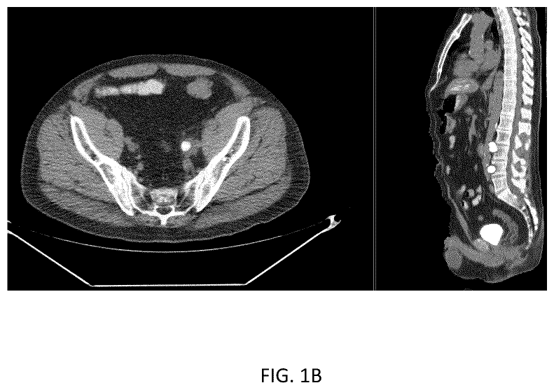

For example, FIG. 1 shows axial slices of a 3D CT image 102 and a 3D PET image 104, along with a fused image 106 in which the slice of the 3D CT image is displayed in grayscale and with the PET image is displayed as a semitransparent overlay. By virtue of the alignment between the CT and PET images, a location of a hotspot within the PET image, indicative of accumulated radiopharmaceutical and, accordingly a potential lesion, can be identified in the corresponding CT image, and viewed in anatomical context, for example, within a particular location in the pelvic region (e.g., within a prostate). FIG. 1B shows another PET/CT fusion, showing a transvers plane slice and a sagittal plane slice.

In certain embodiments, the aligned pair are a composite image, such as a PET/CT or SPECT/CT. In certain embodiments, an anatomical image (e.g., a 3D anatomical image, such as a CT image) and a functional image (e.g., a 3D functional image, such as a PET or SPECT image) are acquired using separate anatomical and functional imaging modalities, respectively. In certain embodiments, an anatomical image (e.g., a 3D anatomical image, such as a CT image) and a functional image (e.g., a 3D functional image, such as a PET or SPECT image) are acquired using a single multimodality imaging system. A functional image and an anatomical image may, for example, be acquired via two scans using a single multimodal imaging system—for example first performing a CT scan and then, second, performing a PET scan—during which a subject remains in a substantially fixed position.

In certain embodiments, 3D boundaries of particular tissue regions of interest can be accurately identified by analyzing 3D anatomical images. For example, automated segmentation of 3D anatomical images can be performed to segment 3D boundaries of regions such as particular organs, organ sub-regions and soft-tissue regions, as well as bone. In certain embodiments, organs such as a prostate, urinary bladder, liver, aorta (e.g., portions of an aorta, such as a thoracic aorta), a parotid gland, etc., are segmented. In certain embodiments, one or more particular bones are segmented. In certain embodiments, an overall skeleton is segmented.

In certain embodiments, automated segmentation of 3D anatomical images may be performed using one or more machine learning modules that are trained to receive a 3D anatomical image and/or a portion thereof, as input, and segment one or more particular regions of interest, producing a 3D segmentation map as output. For example as described in PCT publication WO/2020/144134, entitled “Systems and Methods for Platform Agnostic Whole Body Segmentation,” and published Jul. 16, 2020, the contents of which are incorporated herein by reference in their entirety, multiple machine learning modules implementing convolutional neural networks (CNNs) may be used to segment 3D anatomical images, such as CT images, of a whole body of a subject and thereby create a 3D segmentation map that identifies multiple target tissue regions across a subject's body.

In certain embodiments, for example to segment certain organs where functional images are believed to provide additional useful information that facilitate segmentation, a machine learning module may receive both an anatomical image and a functional image as input, for example as two different channels of input (e.g., analogous to multiple color channels in a color, RGB, image) and use these two inputs to determine an anatomical segmentation. This, multi-channel, approach is described in further detail in U.S. Patent Publication No. US 2021/0334974 A1, entitled “Systems and Methods for Deep-Learning-Based Segmentation of Composite Images,” and published Oct. 28, 2021, the contents of which is hereby incorporated by reference in its entirety.

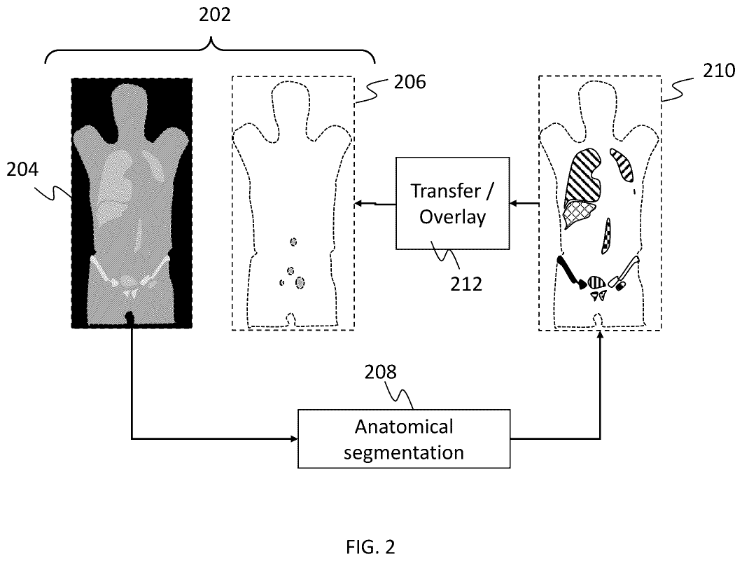

In certain embodiments, as illustrated FIG. 2, an anatomical image 204 (e.g., a 3D anatomical image, such as a CT image) and a functional image 206 (e.g., a 3D functional image, such as a PET or SPECT image) may be aligned with (e.g., co-registered to) each other, for example as in a composite image 202 such as a PET/CT image. Anatomical image 204 may be segmented 208 to create a segmentation map 210 (e.g., a 3D segmentation map) that distinguishably identifies one or more tissue regions and/or sub-regions of interest, such as one or more particular organs and/or bones. Segmentation map 210, having been created from anatomical image 204 is aligned with anatomical image 204, which, in turn, is aligned with functional image 206. Accordingly, boundaries of particular regions (e.g., segmentation masks), such as particular organs and/or bones, identified via segmentation map 210 can be transferred to and/or overlaid 212 upon functional image 206 to identify volumes within functional image 206 for purposes of classifying hotspots, and determining useful indices that serve as measures and/or predictions of cancer status, progression, and response to treatment. Segmentation maps and masks may also be displayed, for example as a graphical representation overlaid on a medical image to guide physicians and other medical practitioners.

### C. Lesion Detection and Characterization

In certain embodiments, approaches described herein include techniques for detecting and characterizing lesions within a subject via (e.g., automated) analysis of medical images, such as nuclear medicine images. Regions of interest (ROIs) in medical images that represent potential lesions may be identified based on, for example, differences in intensity values relative to surroundings, or other characteristic features (e.g., abnormal shapes, texture, spatial frequencies, etc., depending on particular imaging modality). In particular, as described herein, in certain embodiments, hotspots are localized (e.g., contiguous) regions of high intensity, relative to their surroundings, within images, such as 3D functional images and may be indicative of a potential cancerous lesion present within a subject.

A variety of approaches may be used for detecting, segmenting, and classifying hotspots. In certain embodiments, hotspots are detected and segmented using analytical methods, such as filtering techniques including, but not limited to, a difference of Gaussians (DoG) filter and a Laplacian of Gaussians (LoG) filter. In certain embodiments, hotspots are segmented using a machine learning module that receives, as input, a 3D functional image, such as a PET image, and generates, as output a hotspot segmentation map (a “hotspot map”) that differentiates boundaries of identified hotspots from background. In certain embodiments, each segmented hotspot within a hotspot map is individually identifiable (e.g., individually labelled). In certain embodiments, a machine learning module used for segmenting hotspots may take as input, in addition to a 3D functional image, one or both of a 3D anatomical image (e.g., a CT image) and a 3D anatomical segmentation map. The 3D anatomical segmentation map may be generated via automated segmentation (e.g., as described herein) of the 3D anatomical image.

In certain embodiments, segmented hotspots may be classified according to an anatomical region in which they are located. For example, in certain embodiments, locations of individual segmented hotspots within a hotspot map (representing and identifying segmented hotspots) may be compared with 3D boundaries of segmented tissue regions, such as various organs and bones, within a 3D anatomical segmentation map and labeled according to their location, e.g., based on proximity to and/or overlap with particular organs. In certain embodiments, a machine learning module may be used to classify hotspots. For example, in certain embodiments, a machine learning module may generate, as output, a hotspot map in which segmented hotspots are not only individually labeled and identifiable (e.g., distinguishable from each other), but are also labeled, for example, as corresponding to one of a bone, lymph, or prostate lesion. In certain embodiments, one or more machine learning modules may be combined with each other, as well as with analytical segmentation (e.g., thresholding) techniques to perform various tasks in parallel and in sequence to create a final labeled hotspot map.

Various approaches for performing detailed segmentation of 3D anatomical images and identification of hotspots representing lesions in 3D functional images, which may be used with various approaches described herein, are described in PCT publication WO/2020/144134, entitled “Systems and Methods for Platform Agnostic Whole Body Segmentation,” and published Jul. 16, 2020, U.S. Patent Publication No. US 2021/0334974 A1, entitled “Systems and Methods for Deep-Learning-Based Segmentation of Composite Images,” and published Oct. 28, 2021, and PCT publication WO/2022/008374, entitled “Systems and Methods for Artificial Intelligence-Based Image Analysis for Detection and Characterization of Lesions,” and published Jan. 13, 2022, the contents of each of which is incorporated herein in its entirety.

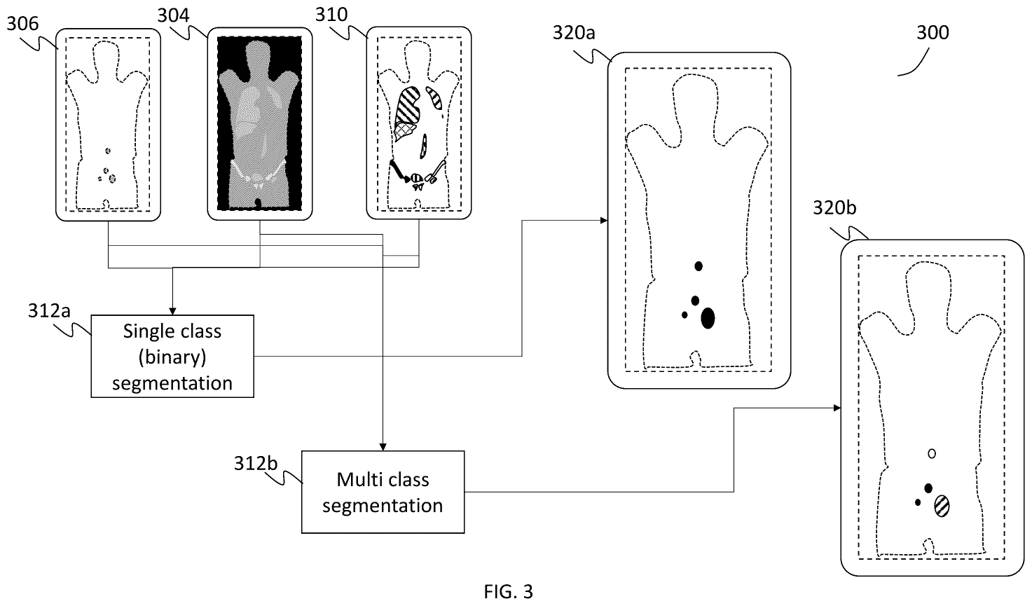

FIG. 3 shows an example process 300 for segmenting and classifying hotspots, based on an example approach described in further detail in PCT publication WO/2022/008374, entitled “Systems and Methods for Artificial Intelligence-Based Image Analysis for Detection and Characterization of Lesions,” and published Jan. 13, 2022. The approach illustrated in FIG. 3 uses two machine learning modules, each of which receives, as input, 3D functional image 306, 3D anatomical image 304, and 3D anatomical segmentation map 310. Machine learning module 312a is a binary classifier that generates a single-class hotspot map 320a, by labeling voxels as hotspot or background (not a hotspot). Machine learning module 312b performs multi-class segmentation, and generates multi-class hotspot map 320b, in which hotspots are both segmented and labeled as one of three classes—prostate, lymph, or bone. Among other things, classifying hotspots in this manner—via a machine learning module 312b (e.g., as opposed to directly comparing hotspot locations with segmented boundaries from segmentation map 310)—obviates a need to segment certain regions. For example, in certain embodiments, machine learning module 312b may classify hotspots as belonging to prostate, lymph, or bone, without a prostate region having be identified and segmented from 3D anatomical image 304 (e.g., in certain embodiments, 3D anatomical segmentation map 310 does not comprise a prostate region). In certain embodiments, hotspot maps 320a and 320b are merged, for example by transferring labels from multi-class hotspot map 320b to the hotspot segmentations identified in single-class hotspot map 320a (e.g., based on overlap). Without wishing to be bound to any particular theory, it is believed that this approach combines improved segmentation and detection of hotspots from single class machine learning module 312a with classification results from multi-class machine learning module 312b. In certain embodiments, hotspot regions identified via this final, merged, hotspot map are further refined, using an analytical technique such as an adaptive thresholding technique described in PCT publication WO/2022/008374, entitled “Systems and Methods for Artificial Intelligence-Based Image Analysis for Detection and Characterization of Lesions,” and published Jan. 13, 2022.

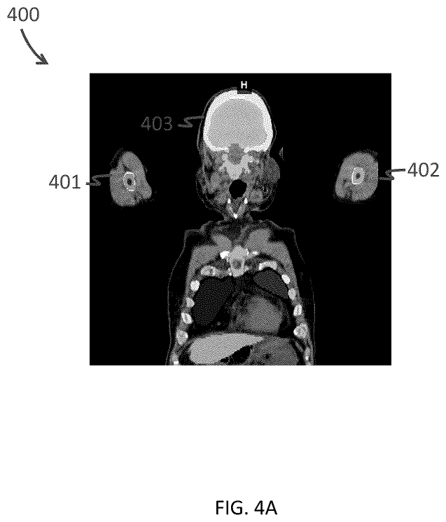

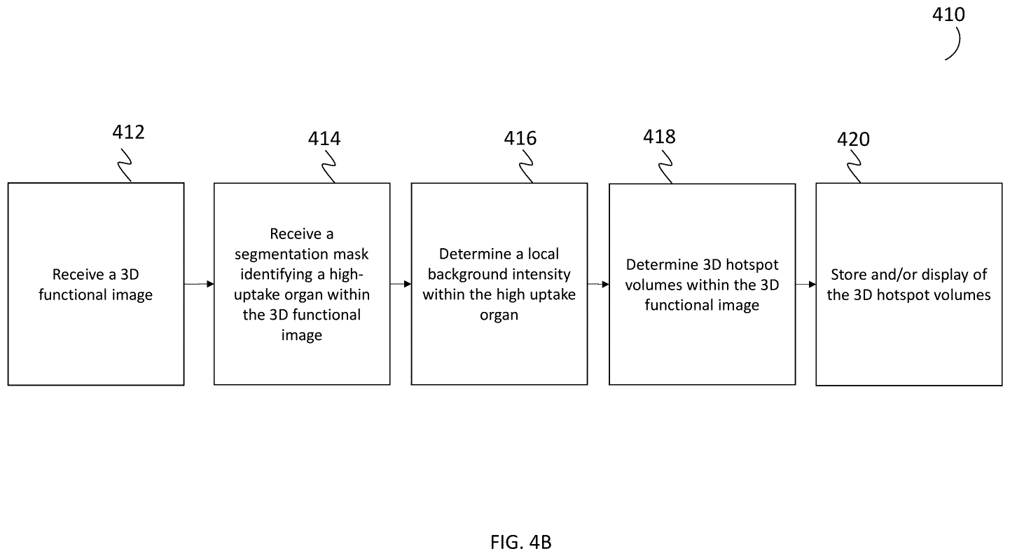

In certain embodiments, once detected and segmented, hotspots may be identified and assigned labels according to a particular anatomical (e.g., tissue) region in which they are located and/or a particular lesion sub-type that they are likely to represent. For example, in certain embodiments, hotspots may be assigned an anatomical location that identifies them as representing locations with a one of a set of tissue regions, such as the listed in Table 1, below. In certain embodiments, a list of tissue regions may include those in Table 1 as well as a gluteus maximus (e.g., left and right) and a gallbladder. In certain embodiments, hotspots are assigned to and/or labeled as belonging to a particular tissue region based on a machine learning classification and/or via comparison of their 3D hotspot volume's location and/or overlap with various tissue volumes identified via masks in an anatomical segmentation map. In certain embodiments, a prostate is not segmented. For example, as described above, in certain embodiments, machine learning module 312b may classify hotspots as belonging to prostate, lymph, or bone, without a prostate region having be identified and segmented from 3D anatomical image 304. In certain embodiments, regions such as a head and/or one or more arm(s) of a patient, may be segmented, for example as shown in FIG. 4A. In certain embodiments, one or more arm(s) comprises upper arm(s) and/or at least a portion of one or both humerus/humeri. In certain embodiments, a head comprises a skull, a mandible, a brain, a parotid gland, and cervical curve, and a cervical vertebra. In certain embodiments, segmentation processes of the present disclosure may utilize a cutoff rule to determine whether to segment a particular region based on an extent of a subject that is imaged, and/or is graphically represented in a medical image. For example, in certain embodiments, if a medical image does not cover (e.g., certain percentage of a region of interest, reach set anatomical landmarks), segmentation of a particular region (e.g., a skull, a parotid gland) is not performed, or may be performed, but discarded or flagged (e.g., as low confidence/potentially erroneous).

In certain embodiments, technologies of the present disclosure provide users with tools that include segmentation procedures that utilize particular, processes for detecting and/or segmenting hotspots that are tailored to different regions (e.g., organs). For example, a user may, seek to locate hotspots in an organ with high physiological uptake, such as a liver that an approach used for (detecting and/or segmenting hotspots within) other organs may struggle with. Accordingly, a tailored approach for detecting and/or segmenting hotspots in high uptake organs may be used.

In certain embodiments, a hotspot segmentation procedure for organ with high physiological uptake may include one or both of: (1) detecting hotspots by applying a model (e.g., statistical, machine-learning) to determine and remove (e.g., filter out) intensities corresponding to normal, background, uptake within the organ; and (2) segmenting hotspots by applying a model (e.g., statistical, machine-learning) to identify and delineate 3D hotspot volume boundaries using the organ boundaries (e.g., which may be identified via one or more segmentation masks determined via approaches described herein, for example, in section B above) and/or determined local background intensity value associated with and representing normal background uptake within the organ (e.g., by detecting abnormally high intensity peaks).

FIG. 4B shows an example process 410 for detecting hotspots in a high uptake organ according to various embodiments described herein. A 3D functional image may be received and/or accessed 412, for example retrieved from memory, either locally or on a PACS server, cloud, etc. A segmentation mask identifying a high uptake organ within the 3D functional image may be received and/or accessed as well 414. A segmentation mask, for example, may be produced from a medical image by automated image processing, manual selection and delineation by a user (e.g., a radiologist), or combinations thereof. For example, various approaches for automated segmentation of anatomical organs and tissue regions, described herein in Section B, may be used to determine a segmentation mask. Using organ boundaries of the high uptake organ from the segmentation mask, a local background intensity within the high uptake organ can be determined 416 from the 3D functional image. Using the determined local background intensity, 3D hotspot volumes within the 3D functional image corresponding to the high uptake organ may be determined 418. Hotspots may be further rendered and displayed 420, for example, as graphical shapes overlaid on medical images.

In certain embodiments, a local background intensity value associated with background uptake in a high-uptake organ comprises is determined as a mean and/or a standard deviation of the uptake value in the organ. In certain embodiments, a local background intensity value is determined by fitting a multi-component mixture model to intensities of voxels within a VOI corresponding to the high uptake organ. For example, a mean and a standard deviation of local background intensity in an organ is determined by fitting a two component Gaussian Mixture model to the organ intensity values. For example, a two component Gaussian mixture model may have the following form:

$$f ( x ) = {\sum }_{i = 1}^{2} {w}_{i} G ( x ; {μ}_{i} , {σ}_{i} )$$

where μᵢ is the mean, σᵢ is the standard deviation and wᵢ is the weight of the i^{th} component and G is the Gaussian function. In certain embodiments, a mean (μ) and a standard deviation (σ) of a component with the largest weight, wᵢ, (e.g., a major mode) is chosen as an estimate of a mean and a standard deviation, respectively, of local background intensity corresponding to normal background uptake within the organ. A smaller component may be assumed to encapsulate abnormally low uptake (e.g., due to misalignment between a medical image and organ segmentation, and/or due to the cysts in the organ causing abnormally low uptake).

In certain embodiments, sub-regions within the high-uptake organ that represent locations of potential hotspots are detected by thresholding organ intensity values and selecting regions with intensities above a detection threshold detection value. In certain embodiments, a detection threshold detection value is determined based on (e.g., as a function of) the local background intensity value, for example based on a mean and standard deviation of a major mode determined via fitting a multi-component mixture model as described herein (e.g., as th=μ+4σ). In certain embodiments, an initial set of preliminary sub-regions are detected, and filtered via one or more selection criteria. One or more selection criteria may include (1) a degree of overlap (e.g., absolute volume, volumetric percentage) between a given one of the one or more preliminary sub-regions and a region (e.g., 3D volume of interest) of the 3D functional image corresponding to the high-uptake organ; (2) a degree of overlap (e.g., absolute volume, volumetric percentage) between a given one of the one or more preliminary sub-regions and a region (e.g., a 3D volume) of the 3D functional image corresponding to at least one other organ or tissue region [e.g., a neighboring organ, an organ with high physiological uptake, an organ known to “bleed over” (e.g., distort uptake values of a neighboring organ due to a misalignment between a functional image and a segmentation mask) into the high uptake organ, e.g., kidney]; (3 a minimum (e.g., hotspot) volume (e.g., a minimum number of voxels; e.g., a minimum corresponding physical volume) [e.g., such that only preliminary sub-regions having volumes greater than or equal to the minimum (e.g., hotspot) volume are selected] [e.g., wherein the minimum hotspot volume is at least 2 (e.g., 1, 5, 10) voxels] (e.g., to avoid noise induced hotspots).

In certain embodiments, hotspots are segmented by sub-dividing a VOI within the functional image corresponding to the high uptake organ into one or more sub-segments, for example using a watershed algorithm. Intensities within the VOI may be smoothed prior to sub-dividing it and peaks detected and used as basins in the watershed algorithm. Smoothing may avoid detecting noise as peaks. detecting uptake peak values (e.g., SUVmax), and uptake peak locations (e.g., locations of SUVmax) (e.g., to avoid noise induced hotspots). These determined sub-segments may then be compared with the detected sub-regions representing potential hotspots, to identify, for each preliminary sub-region, a subsegment that it overlaps with. A 3D hotspot volume corresponding to a particular sub-region (e.g., a j^{th} sub-region and corresponding hotspot volume) may be segmented using an individual segmentation threshold value (tsⱼ), determined based on (e.g., as a maximum of) the detection threshold value and a measure of intensity within the particular sub-region (e.g., a particular percentage, e.g., 60%, of the SUV max). For example, for a j^{th} sub-region and corresponding 3D hotspot volume to be segmented, a corresponding individual segmentation threshold may be determined via,

$$t {s}_{j} = max ( μ + 4 σ , 0.6 \cdot SUV {max}_{j} ) .$$

In certain embodiments, a 3D hotspot volume is segmented using a flood fill algorithm originating in the SUVmax of the detected hotspot, including values that are above the threshold tsⱼ, and are located in the subsegment(s) that the original sub-region (that represents the detected hotspot) overlaps with (e.g., the hotspot segmentation is not allowed to spread into subsegments that were not previously overlapped when the hotspot was detected).

TABLE 1
Certain Tissue Regions (*Prostate may, optionally,
be segmented if present - may be absent if patient has,
e.g., undergone radical prostatectomy, or may not
segmented in any case, in certain embodiments).
Organs/Bones

Right and Right Lung
Left and Right Femur
Left and Right Hip Bone
Urinary bladder
Sacrum and coccyx
Liver
Spleen
Left and Right Kidney
Left Side and Right Side Ribs 1-12
Left and Right Scapula
Left and Right Clavicle
Cervical vertebrae
Thoracic vertebrae 1-12
Lumbar vertebrae 1-5
Sternum
Aorta, thoracic part
Aorta, abdominal part
Prostate*

In certain embodiments, additionally or alternatively, hotspots may be classified as belonging to one or more lesion sub-types. In certain embodiments, lesion sub-type classifications may be made by comparing hotspot locations with classes of anatomical regions. For example, in certain embodiments a miTNM classification scheme may be used, where hotspots are labeled as belonging to one of three classes—miT, miN, or miM—based on whether they represent lesions located within a prostate (miT), pelvic lymph node (miN), or distant metastases (miM). In certain embodiments, a five class version of the miTNM scheme may be used, with distant metastases further divided into three sub classes—miMb for bone metastases, miMa for lymph metastases, and miMc for other soft tissue metastases.

For example, in certain embodiments, hotspots located within a prostate are labeled as belonging to class “T” or “miT”, e.g., representing local tumor. In certain embodiments, hotspots located outside a prostate, but within a pelvic region are labeled as class “N” or “miN”. In certain embodiments, for example as described in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety, a pelvic atlas may be registered to identify boundaries of a pelvic region and/or various sub-regions therein, for purposes of identifying pelvic lymph node lesions. A pelvic atlas may, for example, include boundaries of a pelvic region and/or a planar reference (e.g., a plane passing through an aorta-bifurcation) which hotspot locations can be compared to (e.g., such that hotspots located outside the pelvic region and/or above the planar reference passing through an aorta bifurcation are labeled as “M” or “miM”—e.g., distant metastases). In certain embodiments, distant metastases may be classified as lymph (miMa), bone (miMb), or visceral (miMc) based on a comparison of hotspot locations with an anatomical segmentation map. For example, hotspots located within one or more bones (e.g., and outside a pelvic region) may be labeled as distant metastases, hotspots located within one or more segmented organs or a subset of organs (e.g., brain, lung, liver, spleen, kidneys) may be labeled as visceral (miMc) distant metastases, and remaining hotspots located outside a pelvic region labeled as distant lymph metastases (miMa).

Additionally or alternatively, in certain embodiments, hotspots may be assigned an miTNM class based on a determination that they are located within a particular anatomical region, for example based on a table such as Table 2, where each column corresponds to a particular miTNM label (first row indicating the particular miTNM class) and includes, in rows two and below, particular anatomical regions associated with each miTNM class. In certain embodiments, a hotspot can be assigned as being located within a particular tissue region listed in Table 2 based on a comparison of the hotspot's location with an anatomical segmentation map, allowing for an automated miTNM class assignment.

TABLE 2
An Example List of Tissue Regions Corresponding to Five
Classes in a Lesion Anatomical Labeling Approach.

| | | Pelvic lymph | | |
|---|---|---|---|---|
| Bone | Lymph nodes | nodes | Prostate | Visceral |
| Mb | Ma | N | T | Mc |
| Skull | Cervical | Template right | Prostate | Brain |
| Thorax | Supraclavicular | Template left | | Neck |
| Vertebrae | Axillary | Presacral | | Lung |
| lumbar | | | | |
| Vertebrae | Mediastinal | Other, pelvic | | Esophageal |
| thoracic | | | | |
| Pelvis | Hilar | | | Liver |
| Extremities | Mesenteric | | | Gallbladder |
| | Elbow | | | Spleen |
| | Popliteal | | | Pancreas |
| | Peri-/para-aortic | | | Adrenal |
| | Other, non- | | | Kidney |
| | pelvic | | | |
| | | | | Bladder |
| | | | | Skin |
| | | | | Muscle |
| | | | | Other |

In certain embodiments, hotspots may be further classified in terms of their anatomical location and/or lesion sub-type. For example, in certain embodiments, hotspots identified as located in pelvic lymph (miN) may be identified as belonging to a particular pelvic lymph node sub-region, such as one of a left/right internal iliac, a left or right external iliac, a left or right common iliac, a left or right obturator, a presacral region, or other pelvic region. In certain embodiments, distant lymph node metastases (miMa) may be classified as retroperitoneal (RP), supradiaphragmatic (SD), or other extrapelvic (OE). Approaches for regional (miN) and distant (miMa) lymph metastases classifications may include registration of pelvic atlas images and/or identification of various whole body landmarks, which are described in further detail in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety.

### D. Individual Hotspot Quantification Metrics

In certain embodiments, detected—e.g., identified and segmented—hotspots may be characterized via various individual hotspot quantification metrics. In particular, for a particular individual hotspot, individual hotspot quantification metrics can be used to quantify a measure of size (e.g., 3D volume) and/or intensity of the particular hotspot in a manner that is indicative of a size and/or level of radiopharmaceutical uptake within the (e.g., potential) underlying physical lesion that the particular hotspot represents. Accordingly, individual hotspot quantification metrics may convey, for example to a physician or radiologist, a likelihood that a hotspot appearing in an image represents a true underlying physical lesion and/or convey a likelihood or level of malignancy thereof (e.g., allowing to differentiate between benign and malignant lesions).

In certain embodiments, image segmentation, lesion detection, and characterization techniques as described herein are used to determine, for each of one or more medical images, a corresponding set of hotspots. As described herein, image segmentation techniques may be used to determine, for each hotspot detected in a particular image, a particular 3D volume—a 3D hotspot volume—representing and/or indicative of a volume (e.g., 3D location and extent) of a potential underlying physical lesion within the subject. Each 3D hotspot volume, in turn, comprises a set of image voxels, each having a particular intensity value.

Once determined, a set of 3D hotspot volumes may be used to compute one or more hotspot quantification metrics for each individual hotspot. Individual hotspot quantification metrics may be computed according to various methods and formulae described herein, for example below. In the description below, the variable L is used to refer to a set of hotspots detected with a particular image, with L={1, 2, . . . , 1, . . . , N_{L}}representing a set of N_{L} (i.e., N_{L} being the number of hotspots) hotspots detected within an image and the variable l indexing the l^{th} hotspot. As described herein, each hotspot corresponds to a particular 3D hotspot volume within an image, with Rₗ denoting the 3D hotspot volume of the l^{th} hotspot.

Hotspot quantification metrics may be presented to a user via a GUI and/or a (e.g., automatically or semi-automatically) generated report. As described in further detail herein, individual hotspot quantification metrics may include hotspot intensity metrics and hotspot volume metrics (e.g., lesion volume) that quantify an intensity and size, respectively of a particular hotspot and/or underlying lesion it represents. Hotspot intensity and size may, in turn, be indicative of a level of radiopharmaceutical uptake within, and size of, respectively, an underlying physical lesion within the subject.

D.i. Hotspot Intensity Metrics

In certain embodiments, a hotspot quantification metric is or comprises an individual hotspot intensity metric that quantifies an intensity of an individual 3D hotspot volume. Hotspot intensity metrics may be computed based on individual voxel intensities within identified hotspot volumes. For example, for a particular hotspot, a value of a hotspot intensity metric may be computed as a function of at least a portion (e.g., a particular subset, e.g., all) of that hotspot's voxel intensities. Hotspot intensity metrics may include, without limitation, metrics such as a maximum hotspot intensity, a mean hotspot intensity, and peak hotspot intensity, and the like. As with voxel intensities in nuclear medicine images, in certain embodiments hotspot intensity metrics may represent (e.g., be in units of) SUV values.

In certain embodiments, a value of a particular hotspot intensity metric are computed, for a subject hotspot, based on (e.g., as a function of) that subject hotspot's voxel intensities alone, e.g., and not based on intensities of other image voxels outside the subject hotspot's 3D volume.

For example, a hotspot intensity metric may be a maximum hotspot intensity (e.g., SUV), or “SUV-max,” computed as a maximum voxel intensity (e.g., SUV or uptake) within a 3D hotspot volume. In certain embodiments, a maximum hotspot intensity may be computed according to equations (1a), (1b), or (1c), below

$${Q}_{max} ( l ) = {max}_{i ∈ {R}_{l}} ( {q}_{i} ) \qquad ( 1 a )$$

$$SU {V}_{max} ( l ) = {max}_{i ∈ {R}_{l}} ( {SUV}_{i} ) \qquad ( 1 b )$$

$$SUV = max ( UptakeInVoxel ∈ lesion volume ) \qquad ( 1 c )$$

where, in equations (1a) and (1b) l represents a particular (e.g., l^{th}) hotspot, as described above, qᵢ is the intensity of voxel i and i∈Rₗ is the set of voxels within the particular 3D hotspot volume, Rₗ. In equation (1b), SUVᵢ indicates a particular unit—standardized uptake value (SUV)—of voxel intensity, as described herein.

In certain embodiments, a hotspot intensity metric may be a mean hotspot intensity (e.g., SUV), or “SUV-mean,” and may be computed as a mean over all voxel intensities (e.g., SUV or uptake) within a 3D hotspot volume. In certain embodiments, a mean hotspot intensity may be computed according to equations (2a), (2b), or (2c) below.

$${Q}_{m e a n} ( l ) = {mean}_{i ϵ {R}_{l}} ( {q}_{i} ) = \frac{1}{{n}_{l}} {\sum }_{i ϵ {R}_{l}} {q}_{i} \qquad ( 2 a )$$

$$SU {V}_{m e a n} ( l ) = {mean}_{i ϵ {R}_{l}} ( {SUV}_{i} ) = \frac{1}{{n}_{l}} {\sum }_{i ϵ {R}_{l}} S U {V}_{i} \qquad ( 2 b )$$

$$SU {V}_{m e a n} = {\sum }_{i ∈ lesion volume} \frac{UptakeInVoxel}{{n}_{l}} \qquad ( 2 c )$$

where nₗ is the number of individual voxels within a particular 3D hotspot volume.

In certain embodiments, a hotspot intensity metric may be a peak hotspot intensity (e.g., SUV), or “SUV-peak,” and may be computed as a mean over intensities of the voxels (e.g., SUV or uptake) whose midpoints are located within a (e.g., pre-defined) particular distance (e.g., within 5 mm) of the midpoint of the hotspot voxel where the maximum intensity (e.g., SUV-max) is located within a hotspot, and, accordingly, may be computed according to equations (3a)-(3c) below.

$${Q}_{p e a k} ( l ) = \frac{1}{{n}_{l}} {{\sum }_{i :}}_{dis t ( {i}_{max} , i ) \leq d} {q}_{i} \qquad ( 3 a )$$

$$SU {V}_{p e a k} ( l ) = \frac{1}{{n}_{l}} {{\sum }_{i :}}_{dis t ( {i}_{max} , i ) \leq d} {SUV}_{i} \qquad ( 3 b )$$

$$SU {V}_{p e a k} = \frac{1}{{n}_{l}} {{{\sum }_{i :}}_{dist ( {SUV}_{max} point , i )}}_{\leq 5 m m} {UptakeInVoxel}_{i} \qquad ( 3 c )$$

where i: dist(iₘₐₓ, i)≤d is the set of (hotspot) voxels having a mid-point within a distance, d, from voxel iₘₐₓ, which is the maximum intensity voxel within the hotspot (e.g., Qₘₐₓ(l)=qᵢ₋ₘₐₓ.
D.ii. Lesion Index Metrics

In certain embodiments, a hotspot intensity metric is individual lesion index value that maps an intensity of voxels within a particular 3D hotspot volume to a value on a standardized scale. Such lesion index values are described in further detail in PCT/EP2020/050132, filed Jan. 6, 2020, and PCT/EP2021/068337, filed Jul. 2, 2021, the content of each of which is hereby incorporated by reference in its entirety. Calculation of lesion index values may include calculation of reference intensity values within particular reference tissue regions, such as an aorta portion (also referred to as blood pool) and/or a liver.

For example, in one particular implementation, a first, blood-pool, reference intensity value is determined based on a measure of intensity (e.g., a mean SUV) within an aorta region and a second, liver, reference intensity value is determined based on a measure of intensity (e.g., a mean SUV) within a liver region. As described in further detail, for example in PCT/EP2021/068337, filed Jul. 2, 2021, the content of which is incorporated herein by reference in its entirety, calculation of reference intensities may include approaches such as identifying reference volumes (e.g., an aorta or portion thereof; e.g., a liver volume) within a functional image, such as a PET or SPECT image, eroding and/or dilating certain reference volumes, e.g., to avoid include voxels on the edge of a reference volume, and selecting subsets of reference voxel intensities, based on modeling approaches, e.g., to account for anomalous tissue features, such as cysts and lesions, within a liver. In certain embodiments, a third reference intensity value may be determined, either as a multiple (e.g., twice) of a liver reference intensity value, or based on an intensity of another reference tissue region, such as a parotid gland.

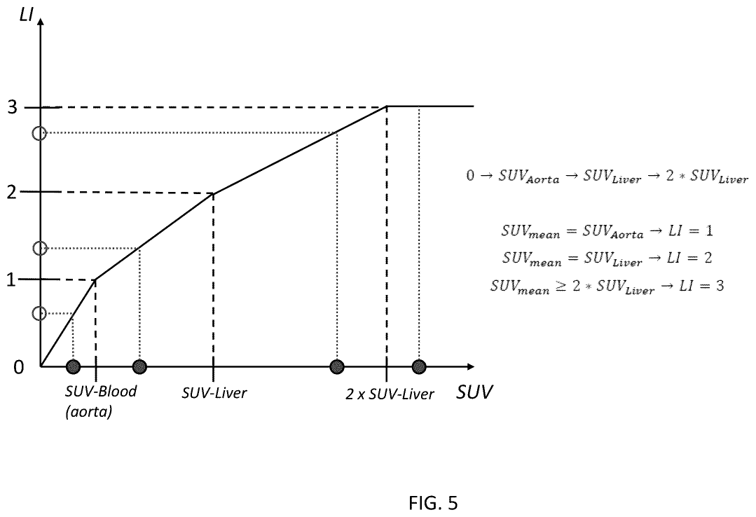

In certain embodiments, hotspot intensities may be compared with one or more reference intensity values to determine a lesion index as a value on a standardized scale, which facilitates comparison across different images. For example, FIG. 4B illustrates an approach for assigning hotspots a lesion index value ranging from 0 to 3. In the approach shown in FIG. 5, a blood-pool (aorta) intensity value is assigned a lesion index of 1, a liver intensity value is assigned a lesion 2, and a value of twice the liver intensity is assigned a lesion index of 3. A lesion index for a particular hotspot can be determined by first computing a value of an initial hotspot intensity metric for the particular hotspot, such as a mean hotspot intensity (e.g., Qₘₑₐₙ(l) or SUVₘₑₐₙ) and comparing the value of the initial hotspot intensity metric with the reference intensity values. For example, the value of the initial hotspot intensity metric may fall within one of four ranges—[0, SUV_{blood}], (SUV_{blood}, SUVₗᵢᵥₑᵣ], (SUVₗᵢᵥₑᵣ, 2×SUVₗᵢᵥₑᵣ], and greater than 2×SUVₗᵢᵥₑᵣ (e.g., (2×SUVₗᵢᵥₑᵣ, ∞)). A lesion index value can then be computed for the particular hotspot based on (i) the value of the initial hotspot intensity metric and (ii) a linear interpolation according to the particular range in which the value of the initial hotspot intensity metric falls, as illustrated in FIG. 5, where the filled and open dots on the horizontal (SUV) and vertical (LI) axes illustrate example values of initial hotspot intensity metrics and resultant lesion index values, respectively. In certain embodiments, if SUV references for either liver or aorta cannot be calculated, or if the aorta value is higher than the liver value, the Lesion Index will not be calculated and will be displayed as ‘-’.

A lesion index value according to the mapping scheme described above and illustrated in FIG. 5 may, for example, be computed as shown in equation (4), below.

$${Q}_{LI} ( l ) = \{ \begin{matrix}{f}_{1} ( S U {V}_{m e a n} ( l ) ) , & SU {V}_{m e a n} ( l ) \leq S U {V}_{aorta} \\ {f}_{2} ( S U {V}_{m e a n} ( l ) ) , & SU {V}_{aorta} \leq S U {V}_{m e a n} ( l ) \leq S U {V}_{l i v e r} \\ {f}_{3} ( S U {V}_{m e a n} ( l ) ) , & SU {V}_{l i v e r} \leq S U {V}_{m e a n} ( l ) \leq 2 \times S U {V}_{l i v e r} \\ 3 , & 2 \times SU {V}_{l i v e r} \leq {SUV}_{m e a n} ( l )\end{matrix} \qquad ( 4 )$$

where f₁ f₂ and f₃ are linear interpolations between the respective spans in equation (4).
D.iii. Hotspot/Lesion Volume

In certain embodiments, a hotspot quantification metric may be a volume metric, such as a lesion volume, Qᵥₒₗ, which provides a measure of size (e.g., volume) of an underlying physical lesion that a hotspot represents. A lesion volume may, in certain embodiments, computed as shown in equations (5a) and (5b), below.

$${Q}_{vol} ( l ) = {\sum }_{i ∈ {R}_{l}} {v}_{i} \qquad ( 5 a )$$

$${Q}_{vol} ( l ) = v \times {n}_{l} \qquad ( 5 b )$$

where in equation (5a), vᵢ is a volume of an i^{th} voxel, and equation (5b) assumes a uniform voxel volume, v, and as before nₗ is a number of voxels in a particular hotspot volume, l. In certain embodiments, a voxel volume is computed as v=δx×δy×δz, where δx, δy, and δz are grid spacing (e.g., in millimeters, mm) in x, y, and z. In certain embodiments, a lesion volume has units of milliliters (ml).

### E. Graphical Widgets for Lesion Selection and Display Control

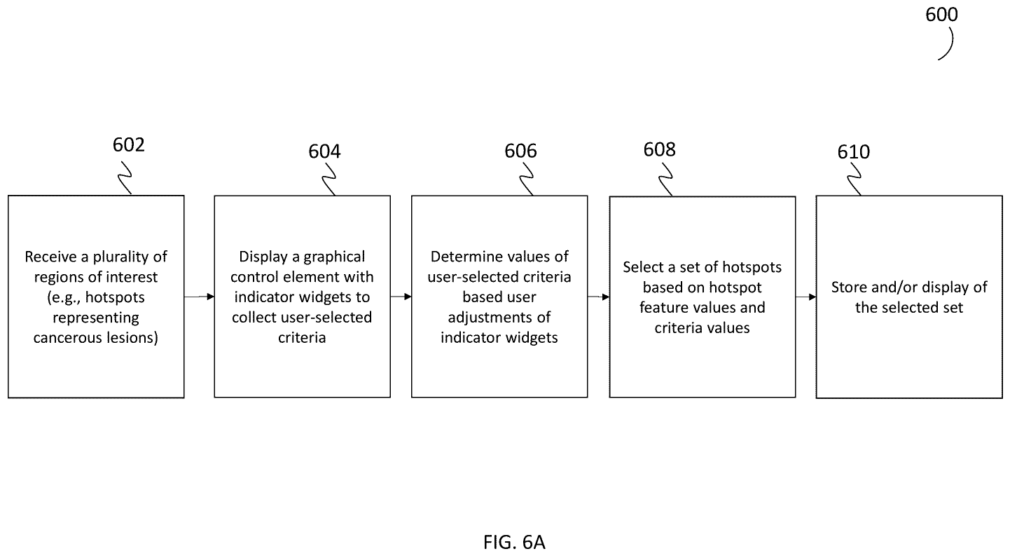

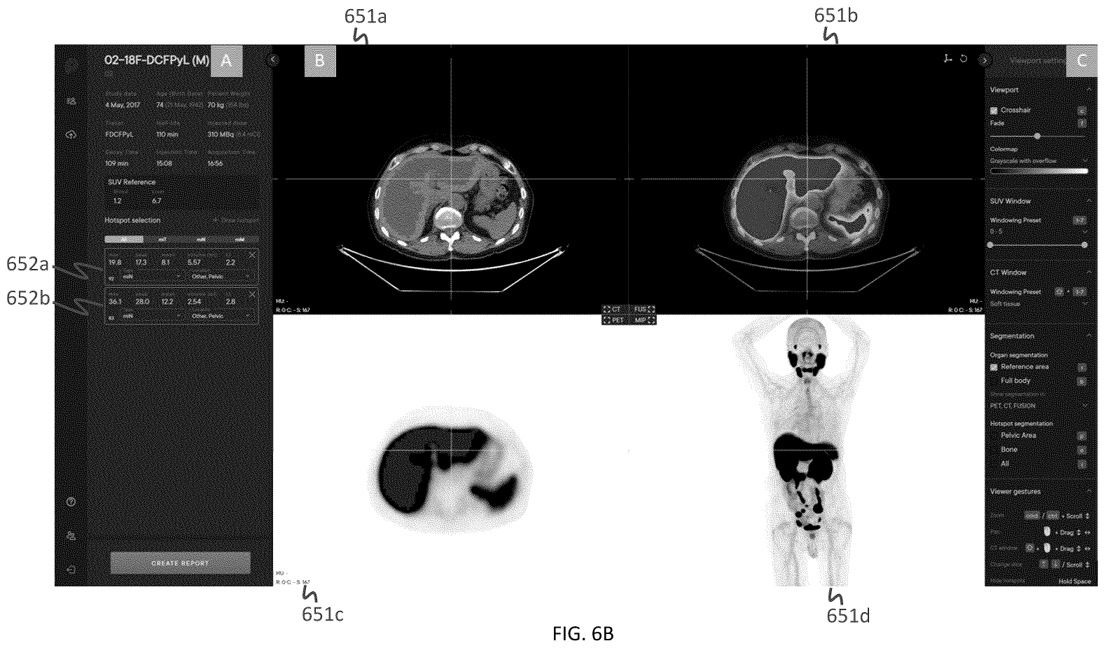

Turning to FIGS. 6A-B, hotspot quantification metrics may be used as hotspot features for sorting and filtering tools, allowing, among other things, users to quickly and efficiently select and focus on (e.g., for review, display, further processing, etc.) subsets of hotspots according to features such as intensity, size, or combinations of both (e.g., lesion indices). Additionally or alternatively, other hotspot features may be used, alone or in combination, to filter/sort detected hotspots. These may be, for example, anatomical region assignments such as those described herein (e.g., in Sections B and C above), lesion classifications (e.g., an miTNM classification, as described in Section C, above), or other features. In certain embodiments, where, for example, hotspots are detected and/or segmented via a machine learning module, likelihood values may be computed for each detected hotspot, and represent a likelihood, e.g., as determined by the machine learning model, that a given detected hotspot represents a true underlying physical lesion within the subject. In certain embodiments, among other things, by allowing a user to filter and/or sort hotspots according to likelihood values, systems and methods of the present disclosures allow users to readily prioritize high or low ‘confidence’ hotspots for their review or other processing.

i. Lesion Selection

FIG. 6A shows an example process 600 for interactive control and selection of hotspots within medical images according to various embodiments described herein. An identification of hotspots may be received and/or accessed 602, for example retrieved from memory, either locally or on a PACS server, cloud, etc. Hotspots identified may be hotspots having been previously detected and delimited in a medical image, by automated image processing, manual selection and delineation by a user (e.g., a radiologist), or combinations thereof. Once accessed/received, as described in further detail herein, hotspots may be rendered and displayed, for example as graphical shapes overlaid on medical images.

Medical images and overlaid hotspots may be displayed for user review and/or analysis within a GUI, for example as shown in FIG. 6B. GUI shown in FIG. 6B allows users to view various views of images, such as PET/CT and/or SPECT/CT images, segmentation maps, and segmented hotspots that represent underlying lesions.

In certain embodiments, once a plurality of hotspots have been (e.g., automatically) detected, a GUI such as shown in FIG. 6B may allow a user to view medical images together with detected hotspots (e.g., overlaid on one or more medical images) from various perspectives and/or different overlays 651a, 651b, 651c, 651d—collectively as a display region 651 of the GUI. In certain embodiments, the GUI shown in FIG. 6B allows a user to select particular hotspots, for example for inclusion in a set of user-selected hotspots and/or to display information about the hotspots.

For example, in certain embodiments, initially (e.g., by default), none of the automatically detected hotspots may be selected, and a user may select individual hotspots, one by one, for inclusion in a set of user-selected hotspots for highlighted display and/or further analysis. A user may, for example, select individual hotspots by clicking on their locations, as displayed as overlays on nuclear medicine (e.g., PET, SPEC) images and/or anatomical images (e.g., CT, X-ray, MRI). In certain embodiments, a processor of a receiving device may receive user selected hotspots and render and/or display them as alphanumeric entries 652a, 652b—collectively hotspot list 652.

In certain embodiments, all of the automatically detected hotspots are selected for inclusion in a set of user-selected hotspots for highlighted display and/or further analysis. In certain embodiments, all of the automatically detected hotspots are displayed as alphanumeric entries 652.

Additionally or alternatively, the GUI shown in FIG. 6B includes graphical control elements that allow a user to select for highlighted display and/or further analysis all automatically detected hotspots having a particular miTNM classification (e.g., miT, miN, miM), and/or to select all hotspots automatically detected, throughout the image.

In certain embodiments, the GUI allows a user to select for highlighted display and/or further analysis all automatically detected hotspots that satisfy each member of a set of one or more user-specified criteria. For example, where the hotspots represent locations of prostate cancer, the set of user-specified criteria may include one or more of the following: (i) tumor/lesion type classification [e.g., miTNM classification, e.g., local tumors (T), regional nodes (N), and/or distant metastases (M)] (e.g., all hotspots having a particular user-specified miTNM classification), (ii) a measure of hotspot intensity, such as a standardized uptake value (SUV) (e.g., all hotspots exceeding a user-identified minimum threshold SUV value), where SUV reflects intensity of the hotspot in relation to a reference intensity level, and (iii) a measure of volume (e.g., all hotspots exceeding a user-identified minimum volume, e.g., a normalized volume). In certain embodiments, the GUI comprises a text box, drop down menu, or other widget for entry and/or selection of alphanumeric character(s) specifying values and/or choices of the one or more user-specified criteria.

In certain embodiments, to select and/or adjust criteria for hotspot selection, users interact with various graphical control elements and indicator widgets, such as sliders, knobs, toggles, radio buttons, check-boxes, drop-down lists, text boxes, and the like, 604. Values of user-selected criteria may, accordingly, be determined based on user adjustment of such indicator widgets 606 and used to select a set of hotspots based on hotspot feature values and criteria values 608. This selected subset may then be stored for display and/or further processing 610.

Among other things, the present disclosure includes the insight that, on the one hand, in certain cases, for example where disease burden is high and a large number of hotspots have been detected, one-at-a-time selection of hotspots can be tedious and time consuming for a user. On the other hand, selecting all hotspots detected in an entire image and/or within particular miTNM classifications can lead to selection excessively large number of hotspots and inclusion of undesired hotspots in a user-selected set. For example, detection approaches, such as machine learning models, used to automatically detect hotspots are often tuned to high sensitivity, so as to be over inclusive, sacrificing positive predictive value—this may be done, for example, especially in the context of medical imaging and cancer detection since a real (medical/human) cost of a false negative (e.g., not detecting a cancerous lesion) can far outweigh that of a false positive.

Accordingly, among other things, in recognition of these aforementioned challenges, systems and methods of the present disclosure provide convenient and user-friendly graphical tools that allow a user to rapidly select hotspots yet, at a same time, maintain fine control over which hotspots are included, even when a large number of hotspots have been detected in high-disease burden cases.

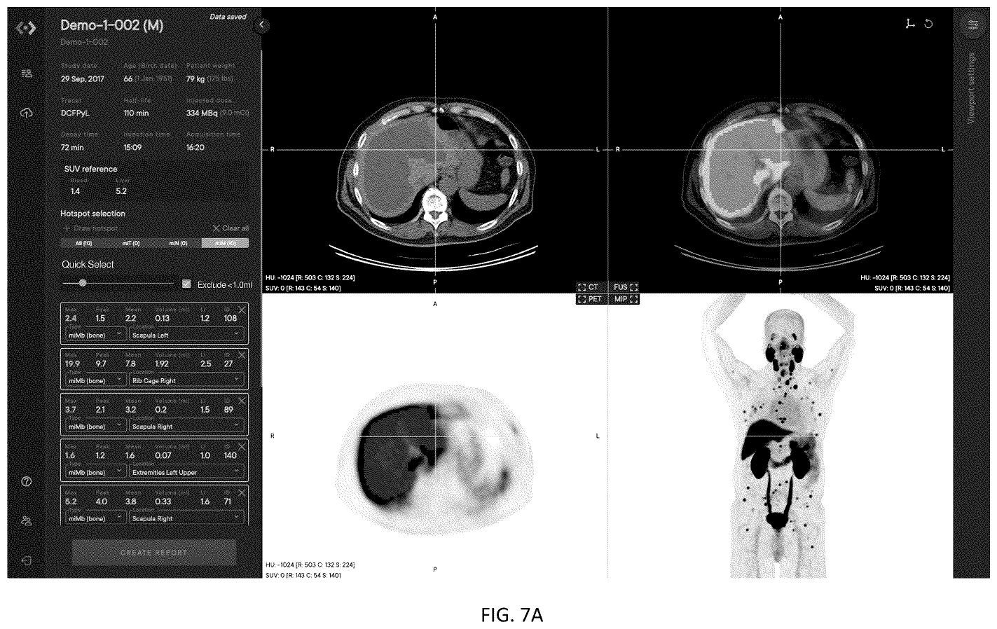

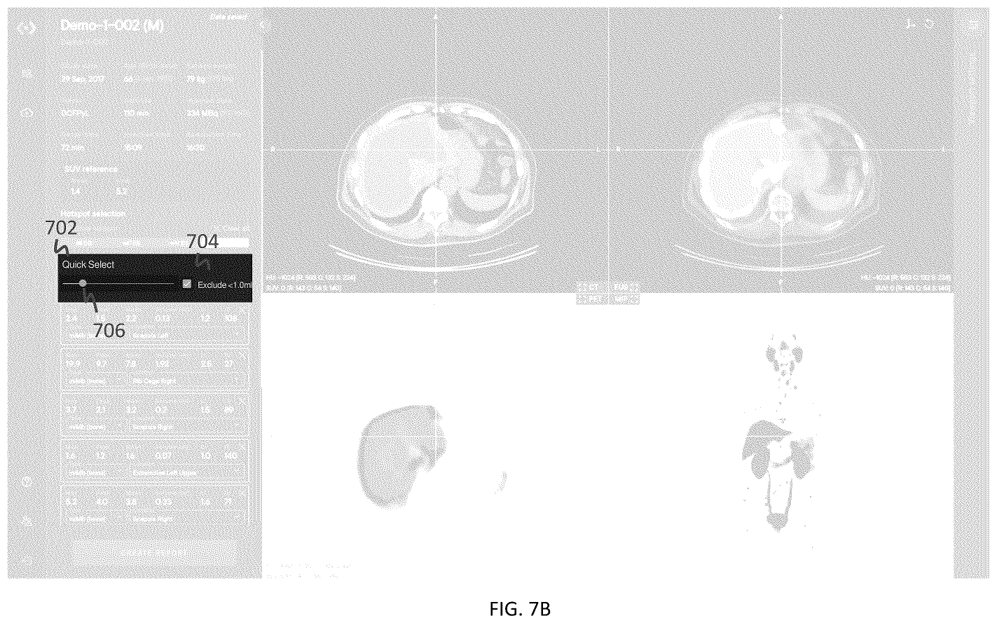

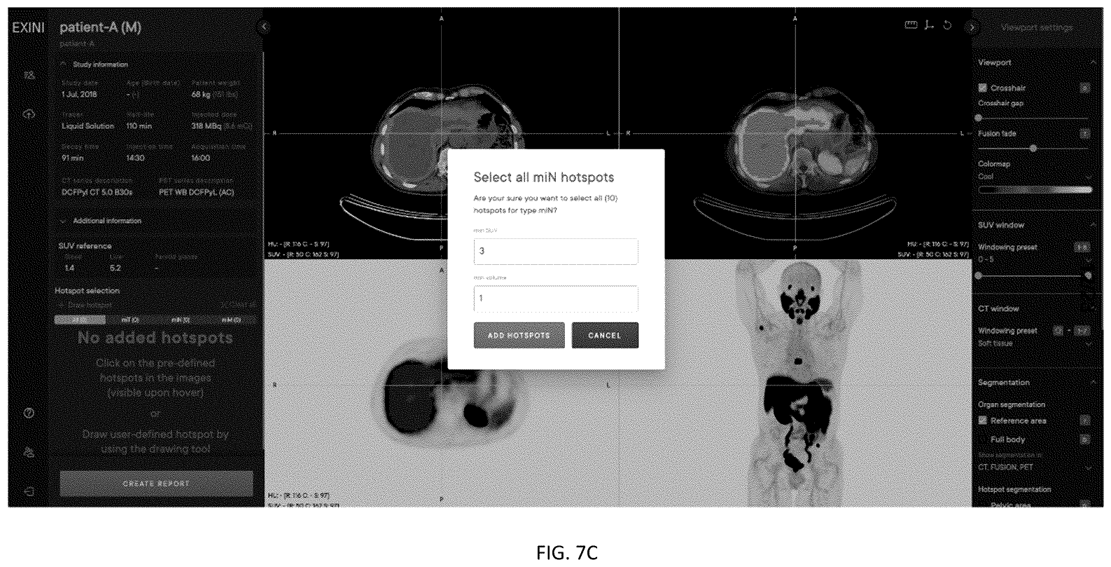

Turning to FIGS. 7A and 7B, systems and methods described herein may include graphical control element, such as a slider 702 or a set of one or more checkboxes 704, which allow for user selection of a subset of a plurality of detected hotspots.

In particular, in certain embodiments, slider 702 may comprise one or more displayed indicator widgets, such as knobs 706 that a user may adjust to control values of one or more thresholds.

For example, in certain embodiments, a plurality of hotspots are detected via a machine learning algorithm such that each is assigned a hotspot likelihood value upon and/or during detection. A hotspot likelihood value may, for example, be a value ranging from zero to one and reflect a certainty/confidence of the machine learning model in its detection of a particular hotspot. In certain embodiments, a likelihood value for a particular hotspot represents a likelihood that the particular detected hotspot represents a true underlying physical lesion (e.g., as opposed to being an image artifact, other feature) (e.g., as calculated by the machine learning model).

In certain embodiments, hotspot likelihood values can be used to assign hotspots positions in an ordered list. Indicator widgets, such as slider knob 706, may then be used to adjust a user-selected rank threshold value, such that hotspots having likelihood values above the user selected rank threshold value are included in a user-selected set, while others are not. In certain embodiments, moving slider knob 706 from one end of scale shown in graphical control element 702 adjusts thresholds so as to select no hotspots at one extreme, and to select all hotspots at another extreme. This ‘quick-select’ approach may be used in addition to (e.g., together with) and/or alternatively (e.g., instead of) one-at-a time selection approaches described herein. For example, in certain embodiments, if hotspots have been selected by clicking, and a user adjusts slider 706, a warning dialog can be shown making a user aware that their current hotspot selection will be overwritten by a ‘quick-select’ slider-based selection.

For example, in certain embodiments, hotspots are assigned in an ordered list of hotspots to slider positions at regular intervals. For example, hotspots with outputs [0.0001, 0.01, 0.3, 0.9] are assigned to [0.25, 0.5, 0.75, 1.0] on a slider with range [0.0, 1.0]. For example, the user may only be interested in ordering, not the distance between hotspots, in terms of output value. The slider gives control over selection in terms of this criteria.

In certain embodiments, hotspot ordering is established for each mi-stage individually. Other metrics such as volume, lesion index, intensity, and the like, may also be used for hotspot ordering.

In certain embodiments, the slider scale is linear—in other embodiments, a non-linear scaling is used for the slider widget. For example, in certain embodiments, the non-linear scaling described in WO 2020/219610 A1, is used.

In certain embodiments, a checkbox 704 or other widget is displayed on the graphical user interface to exclude certain lesions, e.g., lesions that are very small, lesions that are very large, or some other criteria. This selection may be an additional screening (e.g., in addition to the inclusion/exclusion of hotspots based on the slider position) and may be manual, semi-automated, or automated.

Turning to FIG. 7C, systems and methods described herein may include a graphical control element (graphical user interface GUI widget), such as a text box as shown, which allow for user identification of a subset of a plurality of automatically detected hotspots/ROIs for purposes of display and/or further analysis of said subset. The graphical control element is a text box with confirmation button for entry of (i) tumor/lesion type classification [e.g., miTNM classification, e.g., local tumors (T), regional nodes (N), and/or distant metastases (M)] (e.g., all hotspots having a type miN classification), (ii) a measure of hotspot intensity, such as a standardized uptake value (SUV) (e.g., all hotspots exceeding a user-identified minimum threshold SUV value), where SUV reflects intensity of the hotspot in relation to a reference intensity level, and (iii) a measure of volume (e.g., all hotspots exceeding a user-identified minimum volume, e.g., a normalized volume). Upon user entry of the above criteria, the system/method then proceeds with selection, by the processor of the computing device, a subset of the automatically detected hotspot/ROIs based on the ROI feature values and the user selected values of the one or more criteria (e.g., thresholds), then storing and/or providing (e.g., rendering), by the processor, for display and/or further processing, an identification of the user-selected subset (e.g., graphical highlighting of the subset of hotspots).

ii. Lesion Display and Rendering

In certain embodiments, hotspots—e.g., an initial set of e.g., received and/or accessed hotspots, a user selected subset, etc.—may be rendered for graphical display. In certain embodiments, graphical control technologies of the present disclosure provide a user with various options and approaches for controlling rendering of hotspots, among other things, thereby allowing users to view medical images in a convenient and informative manner.

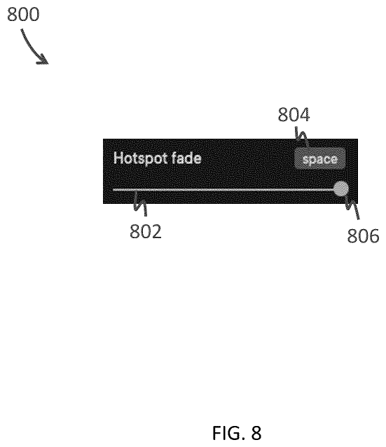

For example, a user may choose to adjust hotspot opacity setting for hotspots displayed in the display region of a GUI as shown in FIG. 8. For example, a user may wish to visualize underlying medical image voxel intensities within and/or neighboring detected hotspots. While hotspots may be rendered as solid-filled graphical shapes, for example making them easy to view in an image, they may obscure underlying medical image voxels. Accordingly, as shown in FIG. 8, a user may adjust an opacity of a solid fill and/or boundary of rendered hotspots, thereby allowing them to visualize underlying medical image voxels in a controllable fashion.

In particular, FIG. 8 shows a schematic of a graphical control element 800 (e.g., a window, a control area, a widget) providing for user adjustment of a hotpot opacity setting. Graphical control element 800 may include a slider 802 and/or a set of one or more checkboxes 804 that allow for user selection of displayed hotspot opacity. Slider 802 may comprise a knob 806, that a user may adjust to control values of the opacity setting. A processor of a computing device may receive opacity settings in this manner via a GUI and render and/or display hotspots according to the received opacity settings (e.g., overlaid on a medical image). In certain embodiments, a processor receives opacity settings only after user confirmation (e.g., via cursor-location-associated mouse clicks, touchpad touches, or other input by the user made within element 800). In certain embodiments, settings are received, and hotspots are rendered and/or displayed according to the received opacity setting in substantially real-time. In certain embodiments, a hotspot opacity setting is applied to all detected hotspots and/or all hotspots of a user selected subset. In certain embodiments, a hotspot opacity setting is applied to an individual hotspot (e.g., selected beforehand, marked by a cursor). In certain embodiments, a hotspot opacity setting applies only to hotspots under a certain category (e.g., tumor/lesion type classification, measure of hotspot intensity, measure of volume). In certain embodiments, a slider regulates hotspot opacity between completely opaque and completely transparent.

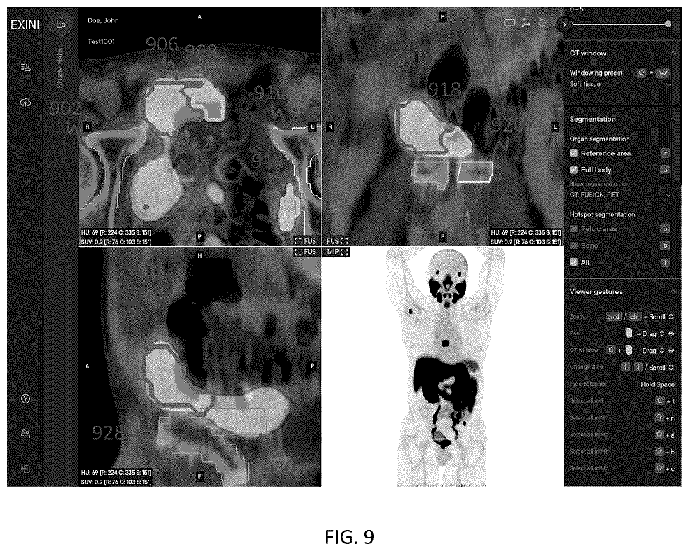

Additionally or alternatively, in certain embodiments, hotspots can be displayed as outlines 902-930 as shown in FIG. 9 and/or shaded regions. In certain embodiments, a user may adjust hotspot outline properties (e.g., color, various thickness, opacity) using a graphical control element (e.g., a window, a control area, a widget). In certain embodiments, a user may adjust display properties of every detected hotspot as a group and/or individually. In certain embodiments, hotspot outlines can be defined by a nuclear image resolution. In certain embodiments, hotspot outlines can be smoothed versions of detected hotspot outlines. Outline smoothing may follow various algorithms of statistics and image processing. In certain embodiments, outline smoothing incorporates a physical model related to imaging properties of imaged tissue and tumors.

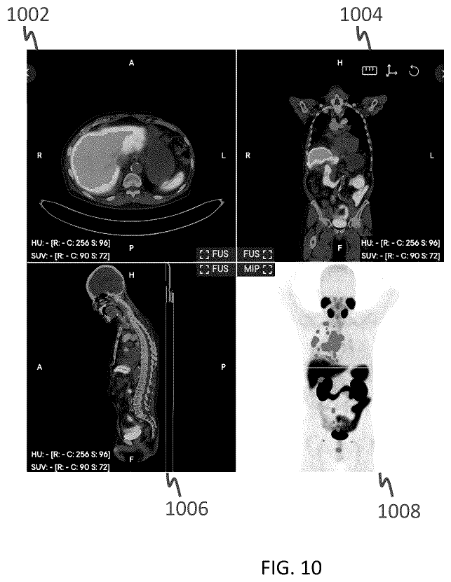

In certain embodiments, a user may choose a particular hanging protocol for displaying medical images in a GUI as shown in FIG. 6B. For example, a user may desire for particular body projections to be displayed for improved image interpretation, for example in the context of particular disease types, stages, medical tasks (e.g., diagnosis, monitoring, staging, treatment planning, treatment review, etc.). In certain embodiments, a user may select a hanging protocol using a dialog box and/or as a selection of one or more entries of a drop-down menu, radio button list, or other graphical user interface element/widget. A processor receives user selection via the GUI and renders and/or displays various projections associated with the selection in the display region of the GUI. In certain embodiments, a hanging protocol may include simultaneous display of axial 1002, coronal 1004, and sagittal 1006 image projections as shown in FIG. 10. In certain embodiments, hanging protocols may comprise axial, sagittal, coronal, multiple intensity, transverse fused projections, multiplane fused (e.g., overlaying a functional and anatomical image), multiplane PET, etc.

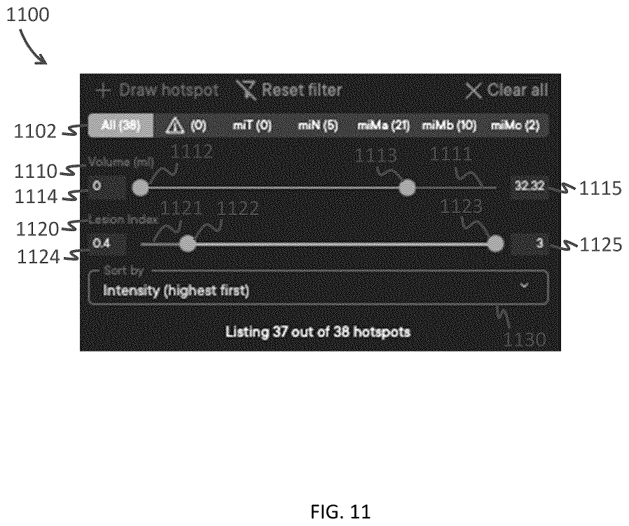

Turning to FIG. 11, in certain embodiments, a user may choose to sort a hotspot list and/or filter hotspots. For example, a user may seek to understand how hotspots compare in terms of various parameters, what hotspots have specific properties, what hotspots miss location assignment, or what properties do hotspots related to specific tissues possess. FIG. 11 shows a schematic of a graphical control element 1100 (e.g., a window, a control area, a widget) within the GUI in which a user selected properties for hotspot filtering. In certain embodiments, the element 1100 may include an element 1102 (e.g., a drop-down menu, a radio button list, or other graphical user interface element/widget) to select hotspots according to a tumor/lesion type classification [e.g., miTNM classification, e.g., local tumors (T), regional nodes (N), and/or distant metastases (M)] (e.g., all hotspots having a type miN classification), an element for selecting a measure of volume 1110 (e.g., all hotspots exceeding a user-identified minimum volume, e.g., a normalized volume), an element for selecting a measure of hotspot intensity, such as lesion index, 1120 (e.g., all hotspots exceeding a user-identified minimum threshold standardized uptake value (SUV), where SUV reflects intensity of the hotspot in relation to a reference intensity level), and a drop-down menu 1130 for selecting sorting order (e.g., from highest to lowest, from lowest to highest, from top to bottom of the body, from bottom to top of the body).

In certain embodiments, the processor receives user-selected properties for hotspot filtering via the GUI, sorts hotspots according to the received selection, and renders and/or displays the sorted hotspots in the display region of the GUI. In certain embodiments, the processor renders and/or displays an alphanumeric list of hotspots with their associated properties. In certain embodiments, the processor receives user selection only after a user confirmation (e.g., via cursor-location-associated mouse clicks, touchpad touches, or other input by the user made within the element 1100 of the GUI). In certain embodiments, the processor receives user selection, renders and/or displays hotspots in the display region of the GUI in real-time.

In certain embodiments, a user may select only one tumor/lesion type in the element 1102. In certain embodiments, a user may select at least one tumor/lesion type in the element 1102. In certain embodiments, the element 1102 comprises selectable options that are associated with alphanumeric markings for tumor/lesion type. In certain embodiments, alphanumeric markings include number of hotspots associated with a tumor/lesion type. In certain embodiments, the element 1102 comprises an unassigned option, where all hotspots that need location assignment are assigned to.

In certain embodiments, the element for selecting a measure of volume 1110 may include a slider 1111. In certain embodiments, the slider 1111 may comprise one or more displayed indicator widgets, such as knobs 1112 and 1113, that a user can adjust to select volume boundaries. In certain embodiments, volume boundaries selected by a user are displayed as alphanumeric indicators 1114 and 1115. In certain embodiments, a user may directly interact with (e.g., edit) indicators 1114 and 1115.

In certain embodiments, the element for selecting a measure of lesion index 1120 may include a slider 1121. In certain embodiments, a slider 1121 may comprise one or more displayed indicator widgets, such as knobs 1122 and 1123, that a user can adjust to select volume boundaries. In certain embodiments, volume boundaries selected by a user are displayed as alphanumeric indicators 1114 and 1115. In certain embodiments, a user may directly interact with (e.g., edit) indicators 1114 and 1115.

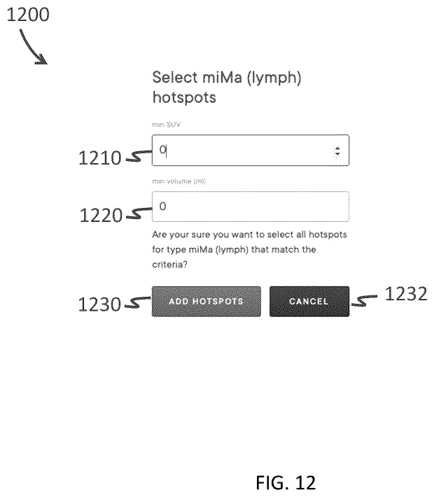

In certain embodiments, a user may select for highlighted display and/or further analysis all automatically detected hotspots having a particular miTNM classification (e.g., miT, miN, miM) and/or other properties (e.g., minimum SUV, minimum volume), throughout the image. For example, a user may seek to quickly add multiple hotspots that would otherwise be labor intensive. FIG. 12 shows a schematic of a graphical control element 1200 (e.g., a window, a control area, a widget) within the GUI in which a user selected hotspot properties to be added. In certain embodiments, element 1200 comprises an input element (e.g., a drop-down menu, a slider, a radio button list, or other graphical user interface element/widget) for minimum hotspot SUV 1210. In certain embodiments, element 1200 comprises an input element (e.g., a drop-down menu, a slider, a radio button list, or other graphical user interface element/widget) for minimum hotspot volume 1220. In certain embodiments, element 1200 comprises a confirmation button 1230 and/or cancellation button 1232.

### F. Tools for Interactive Editing of Lesion Detection and Segmentation

In certain embodiments, technologies of the present disclosure provide users with user-friendly tools that allow them to interactively edit individual hotspot volumes, assign information such as anatomical locations, to hotspot features, and/or rapidly detect and add new hotspot delineations to an existing set of hotspots.

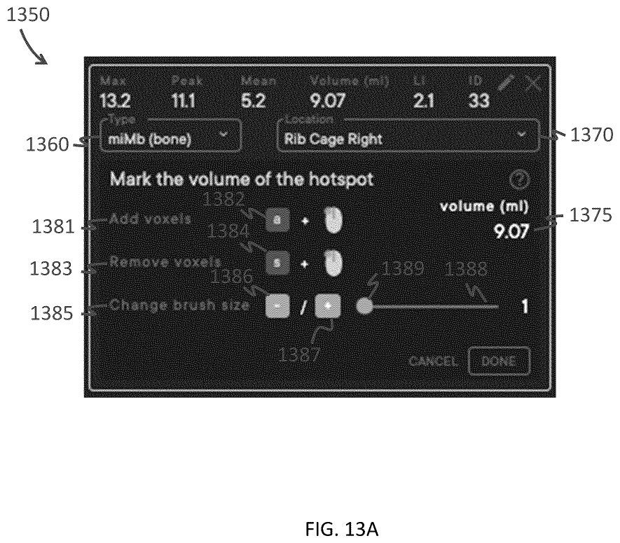

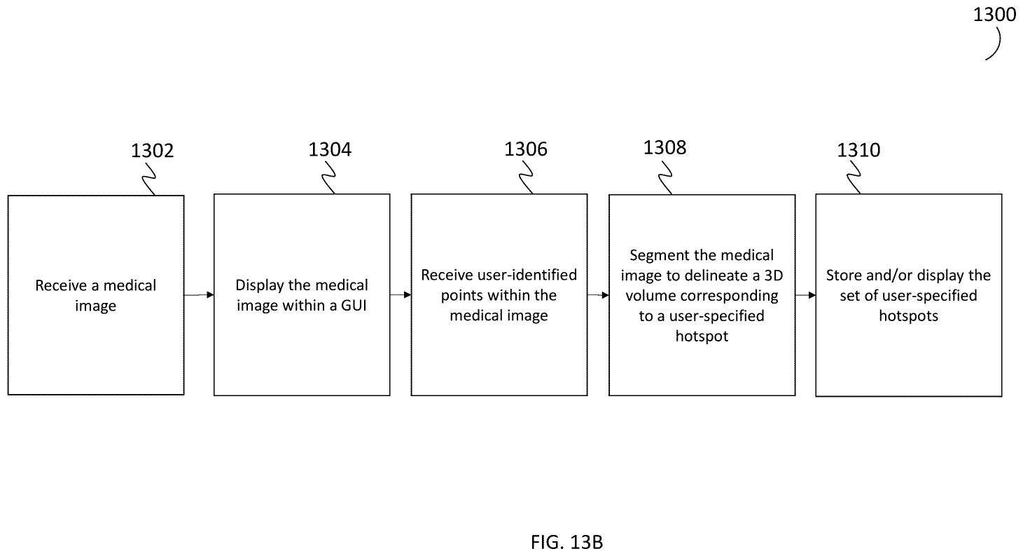

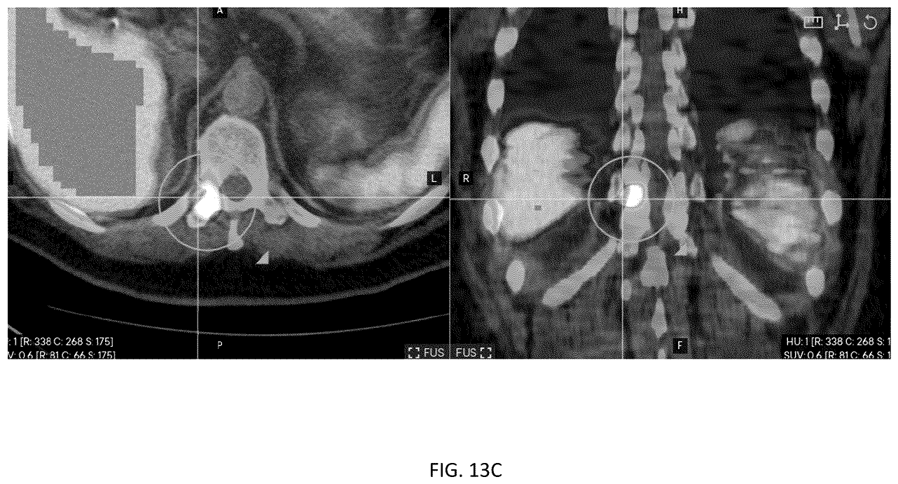

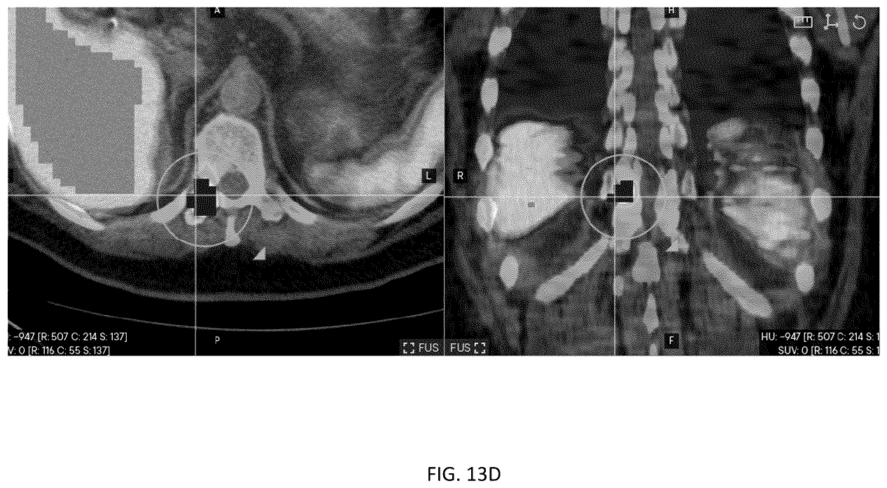

For example, turning to FIG. 13A, a user may wish to edit (e.g., make minor adjustments to) a volume delineated for a particular hotspot, for example to add and/or remove one or more voxels, without the need to completely re-segment or manually mark an entire hotspot. FIG. 13A shows a schematic of a graphical control element 1350 (e.g., a window, a control area, a widget) providing various graphical tools for editing a hotspot volume. In certain embodiments, element 1350 may include an element 1360 (e.g., a drop-down menu, a radio button list, or other graphical user interface element/widget) to select hotspots according to a tumor/lesion type classification [e.g., miTNM classification, e.g., local tumors (T), regional nodes (N), and/or distant metastases (M)] (e.g., all hotspots having a type miN classification), an element 1370 (e.g., a drop-down menu, a radio button list, or other graphical user interface element/widget) to select hotspots location. In certain embodiments, a user may select a hotspot in a display region 651 and its respective properties will be automatically populated in elements 1360 and 1370.

In certain embodiments, a user may add voxels to a hotspot. In certain embodiments, a user may add voxels to a hotspot by directly interacting (e.g., via cursor-location-associated mouse clicks, touchpad touches, or other input by the user) with a displayed medical image and rendered/overlaid hotspot. In certain embodiments, control element 1350 provides instructions (e.g., a combination of mouse clicks and keyboard pressed) to guide a user in adding voxels to a hotspot. In certain embodiments, instructions for adding elements to a hotspot include alphanumeric 1331 and/or pictorial notations 1332.

In certain embodiments, a user may remove voxels from a hotspot. In certain embodiments, a user may remove elements from a hotspot by directly interacting (e.g., via cursor-location-associated mouse clicks, touchpad touches, or other input by the user made within) with a displayed medical image and rendered/overlaid hotspot. In certain embodiments, control element 1350 provides instructions (e.g., a combination of mouse clicks and keyboard pressed) to guide a user in removing voxels from a hotspot. In certain embodiments, instructions for removing voxels from a hotspot include alphanumeric 1383 and/or pictorial 1384 notations.

In certain embodiments, a user may adjust a size of elements (e.g., voxels) to add to and/or remove from a hotspot during a single interaction with a hotspot rendering in the display region 651 (e.g., brush size of a mouse cursor). In certain embodiments, the control element 1350 may include alphanumeric instructions 1385 on adjusting a brush size. In certain embodiments, the control element 1350 may include buttons to reduce 1386 and/or increase 1387 brush size. In certain embodiments, the control element 1350 may include a slider 1388 to adjust the brush size. In certain embodiments, a slider 1388 may comprise one or more displayed indicator widgets, such as a knob 1389, that a user can adjust to change brush size. In certain embodiments, a smallest brush size is set to 1 that corresponds to a single voxel (e.g., resolution limit of a medical image). In certain embodiments, as a user adjusts a hotspot (e.g., by adding elements, removing elements), the processor automatically and in real-time recalculates hotspot volume and renders and/or displays it 1375.

Turning to FIG. 13B, in certain embodiments, a user may manually identify and add hotspots to a set of detected hotspots associated with a medical image. Technologies of the present disclosure allow a user to manually identify and add hotspots via selection of single points within or in a vicinity of user-identified desired hotspots, but otherwise automatically segment the medical image to delineate a 3D boundary/volume of the hotspots. In this manner, users can interactively and accurately identify and delineate 3D hotspot volumes with a single-click. FIG. 13B shows an example process 1300 for single-click detection and segmentation of hotspots. As shown in FIG. 13B, a medical image is received 1302 and displayed 1304 for a user review within a GUI. A user may select individual points within the medical image, which are, in turn, received 1306 and used to segment a 3D volume corresponding to a user specified hotspot 1308, which may, along with other user-specified hotspots, be stored and/or displayed 1310.

Turning to FIG. 13C, in certain embodiments, a user may initiate segmentation of an area by directly interacting (e.g., via cursor-location-associated mouse clicks, touchpad touches, or other input by the user made within) as shown in FIG. 13C. In certain embodiments, a processor receives a user selected position, performs segmentation within a specific volume (e.g., defined by pre-defined boundaries, tissue boundaries), and renders and/or displays the segmented hotspot as shown in FIG. 13D. Accordingly, this single-click approach allows a user to accurately segment hotspots in a semi-automated fashion, allowing a user to manually define at least a portion (e.g., one point) of a hotspot, while automatically carrying out a full segmentation of a hotspot volume. In certain embodiments, a volume for segmentation may be bound within a sub-region of a medical image, such as a spherical volume as shown via the circular indicator in FIGS. 13C and 13D. In certain embodiments, a sub-region is user defined (e.g., via a drop-down menu, a slider, a radio button list, or other graphical user interface element/widget). In certain embodiments, a sub-region for segmentation may be bound by a threshold that is user configured (e.g., via a drop-down menu, a slider, a radio button list, or other graphical user interface element/widget). In certain embodiments, a threshold may be associated with SUV values.

In certain embodiments, single-click hotspot segmentation uses an iterative threshold-based approach, whereby an initial threshold is determined based on an intensity of a point (e.g., a voxel, an average, mean, median etc. of surrounding voxels) selected by the user and then used to segment an initial connected component (region) within the medical image. A local intensity maximum is then determined within the initial connected component region, and used to determine an updated intensity threshold, which is, in turn, used to re-segment the medical image to create a new, updated connected component region. The local intensity maximum is updated based on this new, current, connected component region. In certain embodiments, these two steps, each performed once, are sufficient for segmenting a hotspot. In certain embodiments, the process repeats, iteratively, for example for a fixed number of iterations and/or until a desired stop criteria is met).

An example set of steps for performing a single-click hotspot segmentation as illustrated in FIGS. 13C-13D is as follows:

1. A user defines a spherical region in the medical image.

2. A processor determined an SUV value from a center of the selected region and uses it as a threshold value.

3. The processor performs threshold filtering of the medical image using the SUV value and determines a component that overlaps with the spherical region.

4. The processor determines SUV max, a maximum value of SUV within the component.

5. The processor performs threshold filtering of the medical image using the SUV max value and determines a hotspot, the hotspot is a component that overlap with the spherical region.

6. The processor displays the as-determined hotspot.

In certain embodiments, intensity thresholds are determined from local intensity maxima (or, e.g., at a first iteration, a user selected point) via a thresholding function. In certain embodiments, for example, a thresholding function may determine an intensity threshold as a particular fraction (e.g., a percentage) of a local intensity maxima (e.g., 90% of a local SUV max). In certain embodiments, a thresholding function may determine an intensity threshold based on (i) a local intensity maxima and (ii) one or more reference intensity values, each associated with and determined using intensities of voxels within a corresponding reference tissue region (e.g., a liver; e.g., an aorta portion; e.g., parotid). In certain embodiments, a thresholding function determines an intensity threshold using a linearly decreasing percentage of a local SUV max until a threshold value reaches twice of that of a blood pool reference (e.g., determined within an aorta portion). In certain embodiments, for example, for very low blood pool reference intensities, a thresholding function is determined using a linearly decreasing percentage of a local SUV max until a threshold value reaches 1.1 of that of the blood pool reference.

Example pseudo-code for a thresholding function that uses a blood pool reference is shown below:

```
threshold_function(suv_max, blood_pool):
low_background_level = blood_pool
high_background_level = 2 * max(blood_pool, 1.1)
max_percentage = 90
min_percentage = 50
max_prop = max_percentage / 100
min_prop = min_percentage / 100
suv_start_interpol = low_background_level / max_prop
suv_end_interpol = high_background_level / min_prop
if suv_max <= suv_start_interpol:
  thr = suv_max * max_prop
elif suv_max >= suv_end_interpol:
  thr = high_background_level
else:
  current_prop =
max_prop+(min_prop−max_prop)*(suv_max−
suv_start_interpol)/(suv_end_interpol−suv_start_{—}
interpol)
  thr = suv_max * current_prop
  thr = min(thr, high_background_level) # ensure
monotonicity
return thr
```

### G. Computer System and Network Environment

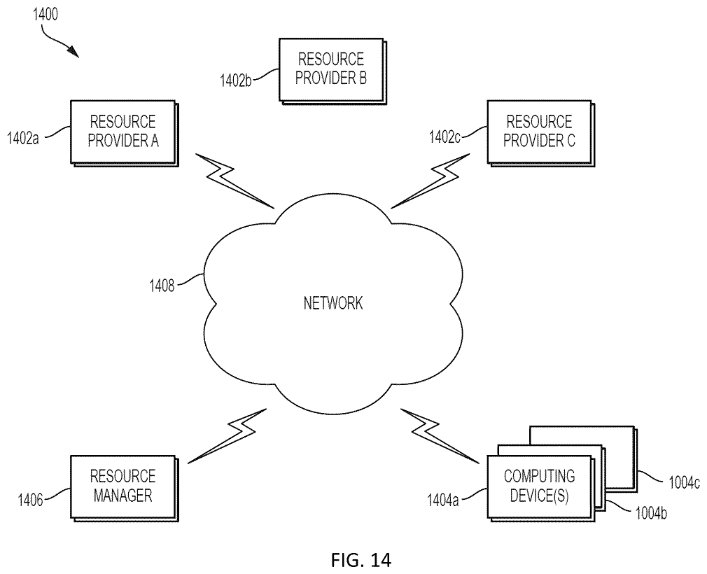

As shown in FIG. 14, an implementation of a network environment 1400 for use in providing systems, methods, and architectures as described herein is shown and described. In brief overview, referring now to FIG. 14, a block diagram of an exemplary cloud computing environment 1400 is shown and described. The cloud computing environment 1400 may include one or more resource providers 1402a, 1402b, 1402c (collectively, 1402). Each resource provider 1402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1402 may be connected to any other resource provider 1402 in the cloud computing environment 1400. In some implementations, the resource providers 1402 may be connected over a computer network 1408. Each resource provider 1402 may be connected to one or more computing device 1404a, 1404b, 1404c (collectively, 1404), over the computer network 1408.

The cloud computing environment 1400 may include a resource manager 1406. The resource manager 1406 may be connected to the resource providers 1402 and the computing devices 1404 over the computer network 1408. In some implementations, the resource manager 1406 may facilitate the provision of computing resources by one or more resource providers 1402 to one or more computing devices 1404. The resource manager 1406 may receive a request for a computing resource from a particular computing device 1404. The resource manager 1406 may identify one or more resource providers 1402 capable of providing the computing resource requested by the computing device 1404. The resource manager 1406 may select a resource provider 1402 to provide the computing resource. The resource manager 1406 may facilitate a connection between the resource provider 1402 and a particular computing device 1404. In some implementations, the resource manager 1406 may establish a connection between a particular resource provider 1402 and a particular computing device 1404. In some implementations, the resource manager 1406 may redirect a particular computing device 1404 to a particular resource provider 1402 with the requested computing resource.

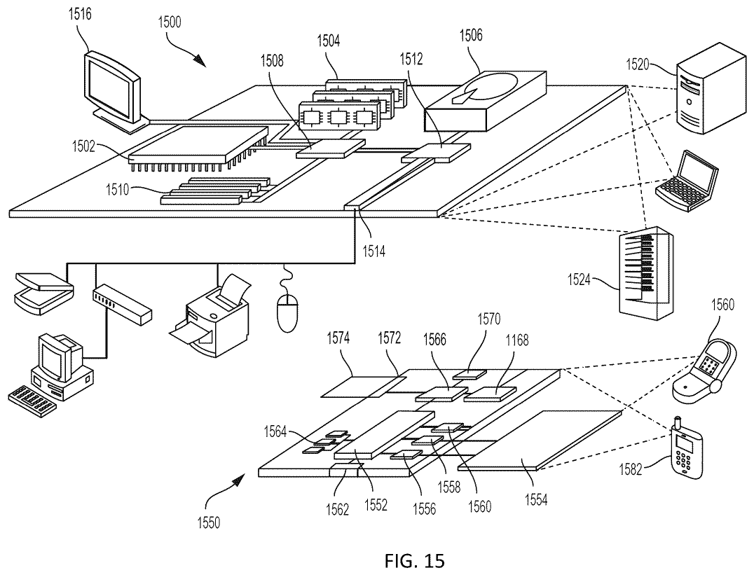

FIG. 15 shows an example of a computing device 1500 and a mobile computing device 1550 that can be used to implement the techniques described in this disclosure. The computing device 1500 is intended to represent various forms of digital computers, such as laptops, desktops, workstations, personal digital assistants, servers, blade servers, mainframes, and other appropriate computers. The mobile computing device 1550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1500 includes a processor 1502, a memory 1504, a storage device 1506, a high-speed interface 1508 connecting to the memory 1504 and multiple high-speed expansion ports 1510, and a low-speed interface 1512 connecting to a low-speed expansion port 1514 and the storage device 1506. Each of the processor 1502, the memory 1504, the storage device 1506, the high-speed interface 1508, the high-speed expansion ports 1510, and the low-speed interface 1512, are interconnected using various busses, and may be mounted on a common motherboard or in other manners as appropriate. The processor 1502 can process instructions for execution within the computing device 1500, including instructions stored in the memory 1504 or on the storage device 1506 to display graphical information for a GUI on an external input/output device, such as a display 1516 coupled to the high-speed interface 1508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1504 stores information within the computing device 1500. In some implementations, the memory 1504 is a volatile memory unit or units. In some implementations, the memory 1504 is a non-volatile memory unit or units. The memory 1504 may also be another form of computer-readable medium, such as a magnetic or optical disk.

The storage device 1506 is capable of providing mass storage for the computing device 1500. In some implementations, the storage device 1506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1502), perform one or more methods, such as those described above. The instructions can also be stored by one or more storage devices such as computer- or machine-readable mediums (for example, the memory 404, the storage device 1506, or memory on the processor 1502).

The high-speed interface 1508 manages bandwidth-intensive operations for the computing device 1500, while the low-speed interface 1512 manages lower bandwidth-intensive operations. Such allocation of functions is an example only. In some implementations, the high-speed interface 1508 is coupled to the memory 1504, the display 1516 (e.g., through a graphics processor or accelerator), and to the high-speed expansion ports 1510, which may accept various expansion cards (not shown). In the implementation, the low-speed interface 1512 is coupled to the storage device 1506 and the low-speed expansion port 1514. The low-speed expansion port 1514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1520, or multiple times in a group of such servers. In addition, it may be implemented in a personal computer such as a laptop computer 1522. It may also be implemented as part of a rack server system 1524. Alternatively, components from the computing device 1500 may be combined with other components in a mobile device (not shown), such as a mobile computing device 1550. Each of such devices may contain one or more of the computing device 1500 and the mobile computing device 1550, and an entire system may be made up of multiple computing devices communicating with each other.

The mobile computing device 1550 includes a processor 1552, a memory 1564, an input/output device such as a display 1554, a communication interface 1566, and a transceiver 1568, among other components. The mobile computing device 1550 may also be provided with a storage device, such as a micro-drive or other device, to provide additional storage. Each of the processor 1552, the memory 1564, the display 1554, the communication interface 1566, and the transceiver 1568, are interconnected using various buses, and several of the components may be mounted on a common motherboard or in other manners as appropriate.

The processor 1552 can execute instructions within the mobile computing device 1550, including instructions stored in the memory 1564. The processor 1552 may be implemented as a chipset of chips that include separate and multiple analog and digital processors. The processor 1552 may provide, for example, for coordination of the other components of the mobile computing device 1550, such as control of user interfaces, applications run by the mobile computing device 1550, and wireless communication by the mobile computing device 1550.

The processor 1552 may communicate with a user through a control interface 1558 and a display interface 1556 coupled to the display 1554. The display 1554 may be, for example, a TFT (Thin-Film-Transistor Liquid Crystal Display) display or an OLED (Organic Light Emitting Diode) display, or other appropriate display technology. The display interface 1556 may comprise appropriate circuitry for driving the display 1554 to present graphical and other information to a user. The control interface 1558 may receive commands from a user and convert them for submission to the processor 1552. In addition, an external interface 1562 may provide communication with the processor 1542, so as to enable near area communication of the mobile computing device 1550 with other devices. The external interface 1562 may provide, for example, for wired communication in some implementations, or for wireless communication in other implementations, and multiple interfaces may also be used.

The memory 1564 stores information within the mobile computing device 1550. The memory 1564 can be implemented as one or more of a computer-readable medium or media, a volatile memory unit or units, or a non-volatile memory unit or units. An expansion memory 1574 may also be provided and connected to the mobile computing device 1550 through an expansion interface 1572, which may include, for example, a SIMM (Single In Line Memory Module) card interface. The expansion memory 1574 may provide extra storage space for the mobile computing device 1550, or may also store applications or other information for the mobile computing device 1550. Specifically, the expansion memory 1574 may include instructions to carry out or supplement the processes described above, and may include secure information also. Thus, for example, the expansion memory 1574 may be provide as a security module for the mobile computing device 1550, and may be programmed with instructions that permit secure use of the mobile computing device 1550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1552), perform one or more methods, such as those described above. The instructions can also be stored by one or more storage devices, such as one or more computer- or machine-readable mediums (for example, the memory 1564, the expansion memory 1574, or memory on the processor 1552). In some implementations, the instructions can be received in a propagated signal, for example, over the transceiver 1568 or the external interface 1562.

The mobile computing device 1550 may communicate wirelessly through the communication interface 1566, which may include digital signal processing circuitry where necessary. The communication interface 1566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1568 using a radio-frequency. In addition, short-range communication may occur, such as using a Bluetooth®, Wi-Fi™, or other such transceiver (not shown). In addition, a GPS (Global Positioning System) receiver module 1570 may provide additional navigation- and location-related wireless data to the mobile computing device 1550, which may be used as appropriate by applications running on the mobile computing device 1550.

The mobile computing device 1550 may also communicate audibly using an audio codec 1560, which may receive spoken information from a user and convert it to usable digital information. The audio codec 1560 may likewise generate audible sound for a user, such as through a speaker, e.g., in a handset of the mobile computing device 1550. Such sound may include sound from voice telephone calls, may include recorded sound (e.g., voice messages, music files, etc.) and may also include sound generated by applications operating on the mobile computing device 1550.

The mobile computing device 1550 may be implemented in a number of different forms, as shown in the figure. For example, it may be implemented as a cellular telephone 1580. It may also be implemented as part of a smart-phone 1582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

### H. Imaging Agents

As described herein, a variety of radionuclide labelled PSMA binding agents may be used as radiopharmaceutical imaging agents for nuclear medicine imaging to detect and evaluate prostate cancer. In certain embodiments, certain radionuclide labelled PSMA binding agents are appropriate for PET imaging, while others are suited for SPECT imaging.

H.i. PET Imaging Radionuclide Labelled PSMA Binding Agents

In certain embodiments, a radionuclide labelled PSMA binding agent is a radionuclide labelled PSMA binding agent appropriate for PET imaging.

In certain embodiments, a radionuclide labelled PSMA binding agent comprises [18F]DCFPyL (also referred to as PyL™; also referred to as DCFPyL-18F):

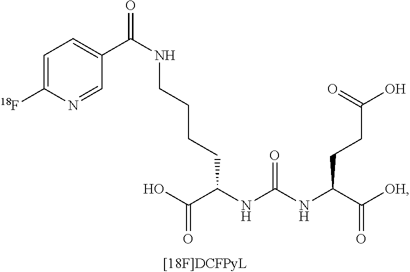 [SMILES: O=C(O)CC[C@H](NC(=O)N[C@@H](CCCCNC(=O)c1ccc([18F])nc1)C(=O)O)C(=O)O]

or a pharmaceutically acceptable salt thereof.

In certain embodiments, a radionuclide labelled PSMA binding agent comprises [18F]DCFBC:

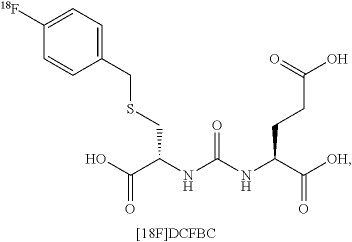 [SMILES: O=C(O)CC[C@H](NC(=O)N[C@@H](CSCc1ccc([18F])cc1)C(=O)O)C(=O)O]

or a pharmaceutically acceptable salt thereof.

In certain embodiments, a radionuclide labelled PSMA binding agent comprises ⁶⁸Ga-PSMA-HBED-CC (also referred to as ⁶⁸Ga-PSMA-11):

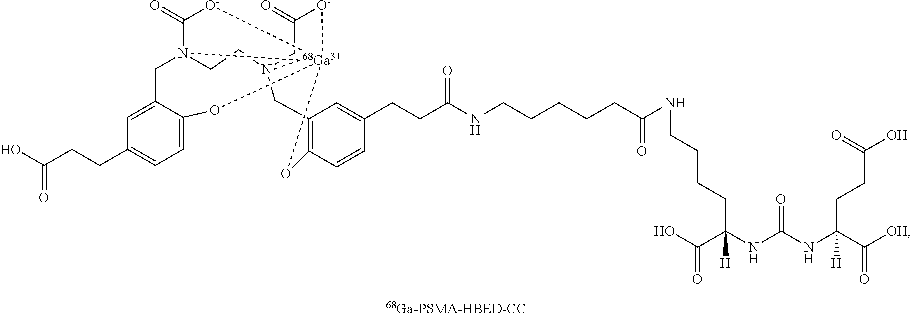

or a pharmaceutically acceptable salt thereof.

In certain embodiments, a radionuclide labelled PSMA binding agent comprises PSMA-617:

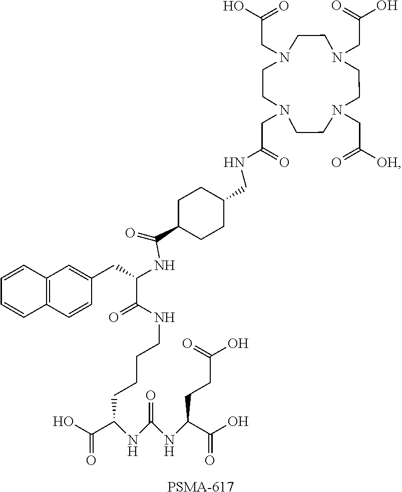

or a pharmaceutically acceptable salt thereof. In certain embodiments, the radionuclide labelled PSMA binding agent comprises ⁶⁸Ga-PSMA-617, which is PSMA-617 labelled with 68Ga, or a pharmaceutically acceptable salt thereof. In certain embodiments, a radionuclide labelled PSMA binding agent comprises ¹⁷⁷Lu-PSMA-617, which is PSMA-617 labelled with 177Lu, or a pharmaceutically acceptable salt thereof.

In certain embodiments, a radionuclide labelled PSMA binding agent comprises PSMA-I&T:

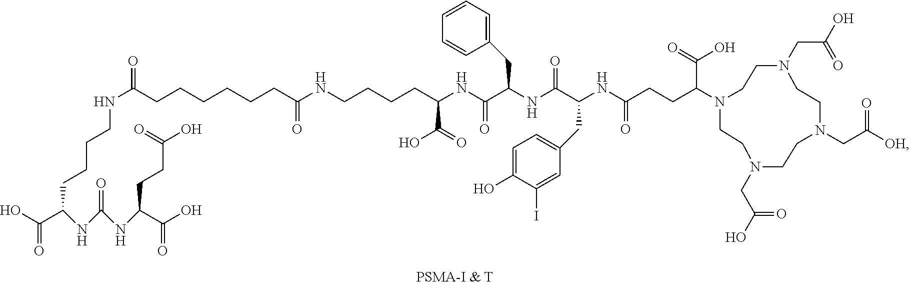

or a pharmaceutically acceptable salt thereof. In certain embodiments, a radionuclide labelled PSMA binding agent comprises ⁶⁸Ga-PSMA-I&T, which is PSMA-I&T labelled with ⁶⁸Ga, or a pharmaceutically acceptable salt thereof.

In certain embodiments, a radionuclide labelled PSMA binding agent comprises PSMA-1007:

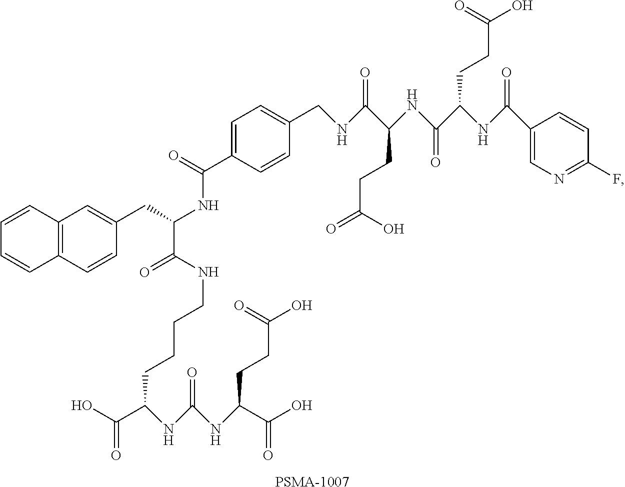

or a pharmaceutically acceptable salt thereof. In certain embodiments, a radionuclide labelled PSMA binding agent comprises ¹⁸F-PSMA-1007, which is PSMA-1007 labelled with ¹⁸F, or a pharmaceutically acceptable salt thereof.

In certain embodiments, a radionuclide labeled PSMA binding agent comprises 18F-JK-PSMA-7:

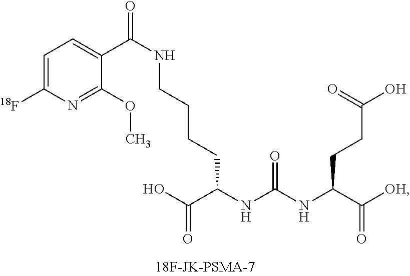 [SMILES: COc1nc([18F])ccc1C(=O)NCCCC[C@H](NC(=O)N[C@@H](CCC(=O)O)C(=O)O)C(=O)O]

or a pharmaceutically acceptable salt thereof.

In certain embodiments, a radionuclide labeled PSMA binding agent comprises (18F) rhPSMA-7.3 (e.g., POSLUMA®):

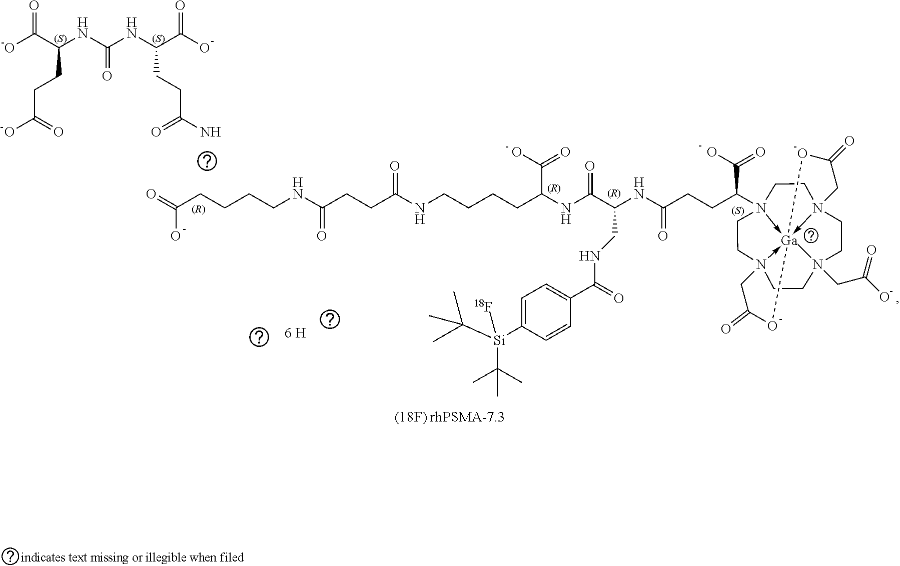

or a pharmaceutically acceptable salt thereof.
H.ii. SPECT Imaging Radionuclide Labelled PSMA Binding Agents

In certain embodiments, a radionuclide labelled PSMA binding agent is a radionuclide labelled PSMA binding agent appropriate for SPECT imaging.

In certain embodiments, a radionuclide labelled PSMA binding agent comprises 1404 (also referred to as MIP-1404):

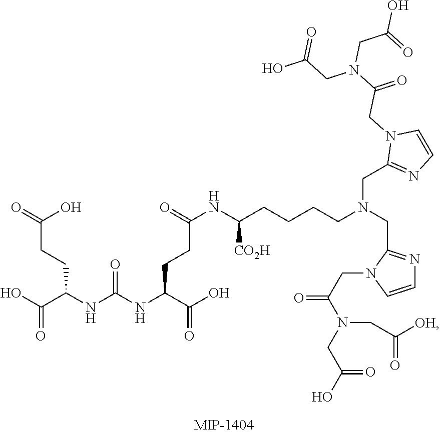 [SMILES: O=C(O)CC[C@H](NC(=O)N[C@@H](CCC(=O)N[C@@H](CCCCN(Cc1nccn1CC(=O)N(CC(=O)O)CC(=O)O)Cc1nccn1CC(=O)N(CC(=O)O)CC(=O)O)C(=O)O)C(=O)O)C(=O)O]

or a pharmaceutically acceptable salt thereof.

In certain embodiments, a radionuclide labelled PSMA binding agent comprises 1405 (also referred to as MIP-1405):

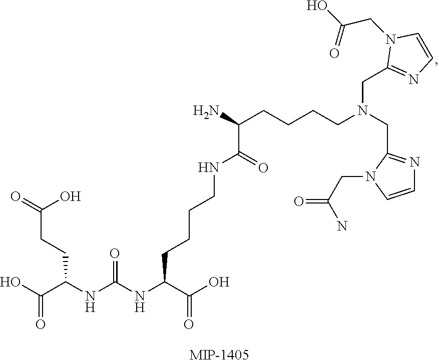 [SMILES: NC(=O)Cn1ccnc1CN(CCCC[C@H](N)C(=O)NCCCC[C@H](NC(=O)N[C@@H](CCC(=O)O)C(=O)O)C(=O)O)Cc1nccn1CC(=O)O]

or a pharmaceutically acceptable salt thereof.

In certain embodiments, a radionuclide labelled PSMA binding agent comprises 1427 (also referred to as MIP-1427):

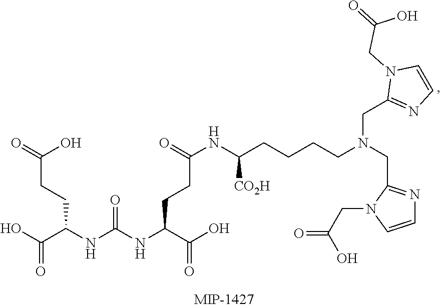 [SMILES: O=C(O)CC[C@H](NC(=O)N[C@@H](CCC(=O)N[C@@H](CCCCN(Cc1nccn1CC(=O)O)Cc1nccn1CC(=O)O)C(=O)O)C(=O)O)C(=O)O]

or a pharmaceutically acceptable salt thereof.

In certain embodiments, a radionuclide labelled PSMA binding agent comprises 1428 (also referred to as MIP-1428):

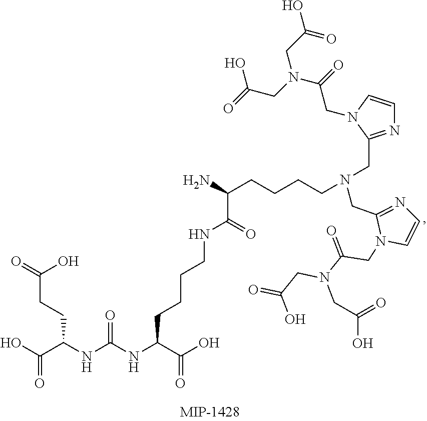 [SMILES: N[C@@H](CCCCN(Cc1nccn1CC(=O)N(CC(=O)O)CC(=O)O)Cc1nccn1CC(=O)N(CC(=O)O)CC(=O)O)C(=O)NCCCC[C@H](NC(=O)N[C@@H](CCC(=O)O)C(=O)O)C(=O)O]

or a pharmaceutically acceptable salt thereof.

In certain embodiments, a PSMA binding agent is labelled with a radionuclide by chelating it to a radioisotope of a metal [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)].

In certain embodiments, 1404 is labelled with a radionuclide (e.g., chelated to a radioisotope of a metal). In certain embodiments, a radionuclide labelled PSMA binding agent comprises ^{99m}Tc-MIP-1404, which is 1404 labelled with (e.g., chelated to)^{99m}Tc:

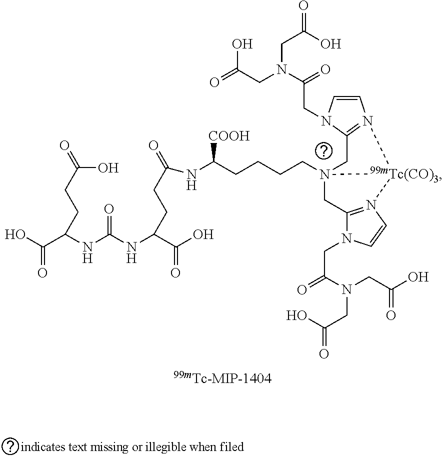

or a pharmaceutically acceptable salt thereof. In certain embodiments, 1404 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1404, with the other metal radioisotope substituted for ^{99m}Tc.

In certain embodiments, 1405 is labelled with a radionuclide (e.g., chelated to a radioisotope of a metal). In certain embodiments, a radionuclide labelled PSMA binding agent comprises ^{99m}Tc-MIP-1405, which is 1405 labelled with (e.g., chelated to)^{99m}Tc:

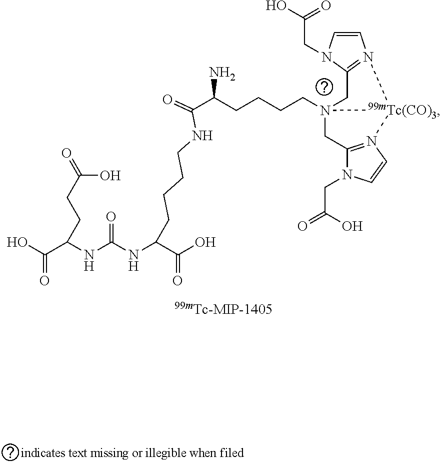

or a pharmaceutically acceptable salt thereof. In certain embodiments, 1405 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1405, with the other metal radioisotope substituted for ^{99m}Tc.

In certain embodiments, 1427 is labelled with (e.g., chelated to) a radioisotope of a metal, to form a compound according to the formula below:

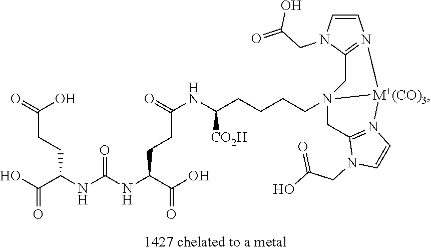

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1427 is labelled.

In certain embodiments, 1428 is labelled with (e.g., chelated to) a radioisotope of a metal, to form a compound according to the formula below:

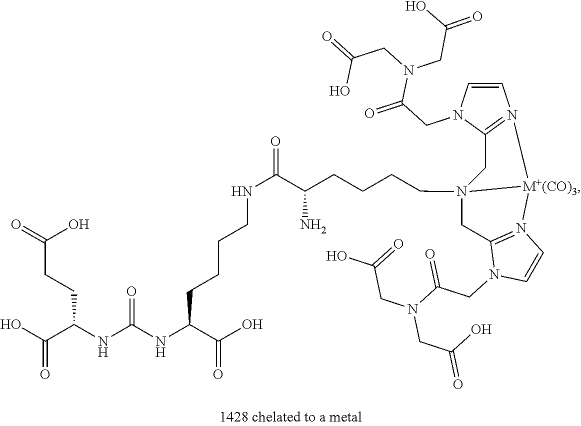

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1428 is labelled.

In certain embodiments, a radionuclide labelled PSMA binding agent comprises PSMA I&S:

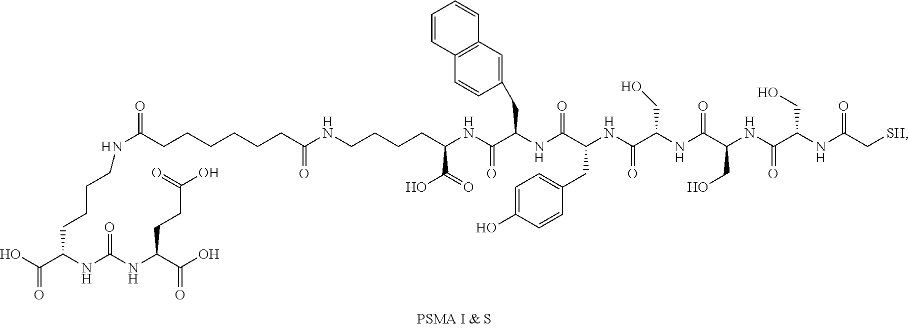 [SMILES: O=C(O)CC[C@H](NC(=O)N[C@@H](CCCCNC(=O)CCCCCCC(=O)NCCCC[C@@H](NC(=O)[C@@H](Cc1ccc2ccccc2c1)NC(=O)[C@@H](Cc1ccc(O)cc1)NC(=O)[C@H](CO)NC(=O)[C@H](CO)NC(=O)[C@H](CO)NC(=O)CS)C(=O)O)C(=O)O)C(=O)O]

or a pharmaceutically acceptable salt thereof. In certain embodiments, a radionuclide labelled PSMA binding agent comprises ^{99m}Tc-PSMA I&S, which is PSMA I&S labelled with ^{99m}Tc, or a pharmaceutically acceptable salt thereof.

### EQUIVALENTS

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1. A method for interactive control of selection and/or analysis of hotspots detected within a medical image of a subject and representing potential lesions, the method comprising:
(a) receiving and/or accessing, by a processor of a computing device, (i) an identification of a plurality of hotspots, and, (ii) a set of hotspot feature values comprising, for each particular hotspot of the plurality of detected hotspots, a corresponding value of at least one hotspot feature representing and/or indicative of a certainty or confidence in detection of the particular hotspot and/or a likelihood that the particular hotspot represents a true physical lesion within the subject;
(b) causing, by the processor, display of a graphical control element allowing for user selection of a subset of the plurality of detected hotspots via user adjustment of one or more displayed indicator widgets within the graphical control element from which values of one or more criteria are determined;
(c) determining, by the processor, based on a user adjustment of the one or more displayed indicator widgets, user selected values of the one or more criteria;
(d) selecting, by the processor, a user-selected subset of the plurality of detected hotspots based on (i) the set of hotspot feature values and (ii) the user selected values of the one or more criteria; and
(e) storing and/or providing, by the processor, for display and/or further processing, an identification of the user-selected subset.

2. The method of claim 1, wherein a particular one of the one or more criteria is a rank threshold whose value corresponds to a position on an ordered list, and the method comprises:
at step (c), determining the value of the rank threshold; and
at step (d), ordering the plurality of hotspots according to their corresponding feature values in the set of hotspot feature values, thereby creating an ordered list of hotspots and selecting, as the user-selected subset, those hotspots having a position in the ordered list of hotspots above and/or below the value of the rank threshold.

3. The method of claim 1, wherein the at least one hotspot feature is or comprises one or more of (i) to (iii) as follows:
(i) a hotspot size that provides a measure of size of a particular hotspot,
(ii) a hotspot intensity that provides a measure of intensity a particular hotspot, and
(iii) an intensity-weighted hotspot size, providing a measure of both size and intensity of a particular hotspot.

4. The method of claim 1, wherein the at least one hotspot feature is or comprises a lesion classification that classifies a given hotspot according to a particular lesion labeling and classification scheme.

5. The method of claim 1, wherein the at least one hotspot feature is or comprises a lesion location identifying an anatomical location of an underlying physical lesion that a given hotspot represents.

6. The method of claim 1, wherein the at least one hotspot feature is or comprises a likelihood value having been determined by the machine learning model upon and/or together with detection of a given hotspot and representing a likelihood, as determined by the machine learning model, that the given hotspot represents a true physical lesion within the subject.

7. The method of claim 1, wherein the one or more criteria comprises one or more of (i)-(iii) as follows:
(i) tumor/lesion type classification,
(ii) a measure of hotspot intensity, and
(iii) a measure of volume.

8. The method of claim 1, wherein the one or more criteria comprise a hotspot likelihood threshold.

9. The method of claim 1, comprising causing, by the processor, graphical rendering of one or both of (i) the plurality of detected hotspots and/or (ii) the user-selected subset of the plurality of detected hotspots, wherein each hotspot is rendered as a graphical shape and/or outline thereof overlaid on the medical image.

10. The method of claim 9, comprising:
causing, by the processor, rendering a plurality of graphical shapes as overlaid on the medical image, each of the plurality of graphical shapes corresponding to and demarking a detected hotspot and having a solid, partially transparent, fill;
receiving, by the processor, via a user interaction with an opacity setting graphical widget, an opacity value; and
updating, by the processor, an opacity of the solid fill and/or boundary of the graphical shapes according to the user-selected opacity value.

11. The method of claim 9, comprising:
causing, the processor, rendering, for each detected hotspot, a graphical outline demarcating a boundary of the hotspot overlaid on the medical image.

12. The method of claim 1, comprising receiving, by the processor, a user selection of a particular hanging protocol and causing, by the processor, display of the medical image according to the particular hanging protocol.

13. The method of claim 1, wherein:
the set of hotspot feature values comprises, for each particular hotspot of the plurality of detected hotspot, a lesion location assignment having an initial value: (i) identifying a particular anatomical region in which the particular hotspot is located or (ii) identifying the particular hotspot has as unassigned;
the one or more user-selected criteria comprise a lesion location assignment criteria; and
the method comprises:
at step (c), determining, via the user interaction with the one or more displayed indicator widgets, as the value of the lesion location assignment criteria, an unassigned hotspots value;
at step (d), selecting, by the as the user-selected subset, all unassigned hotspots;
causing, by the processor, graphical rendering and display of the user-selected subset; and
for each of particular hotspot of at least a portion of the user-selected subset:
receiving, by the processor, a user input of a location assignment for the particular hotspot; and
updating the lesion location assignment for the particular hotspot with the user input location assignment.

14. The method of claim 1, comprising:
receiving, by the processor, a user selection of a particular hotspot of the plurality of detected hotspots;
receiving, by the processor a user selection of one or more voxels of the medical image to add to, and/or subtract from the particular hotspot; and
updating the particular hotspot to incorporate and/or exclude the one or more user-selected voxels.

15. The method of claim 1, wherein at least a portion of the medical image is rendered and display for user viewing and/or review within a graphical user interface (GUI) and the method comprises:
receiving, by the processor, one or more user-identified points within the medical image, each of the one or more user-identified points corresponding to a location of a user single-click within the GUI;
for each particular one of the one or more user-identified points within the medical image, segmenting, by the processor, the medical image to delineate a 3D volume of corresponding user-specified hotspot using (i) the particular user-identified point and (ii) intensities of voxels of the medical image about the particular user-identified point, thereby determining one or more user-specified hotspots, each associated with and segmented via a user single-click; and
updating, by the processor, the initial set of hotspots to include the plurality of user-specified hotspots.

16. The method of claim 15, wherein, for each particular one of the one or more user-identified points within the medical image, segmenting the medical image to delineate the 3D volume of the corresponding user-specified hotspot comprises:
determining a local intensity threshold value based on the intensities of the voxels of the medical image about the particular user-identified point; and
using the local intensity threshold value to segment the medical image to delineate the 3D volume of the corresponding user-specified hotspot.

17. The method of claim 15, wherein for each particular one of the one or more user-identified points within the medical image, segmenting the medical image to delineate the 3D volume of the corresponding user-specified hotspot comprises:
determining an initial intensity threshold value using the particular user-identified point;
at a first step, using the initial intensity threshold value to segment the medical image to identify and delineate a connected region within the medical image and determining an updated intensity threshold value based on intensities of the medical image within the connected region; and
at a second step, segmenting the medical image to identify and delineate an updated connected region updating the intensity threshold value based on intensities of the medical image within the updated connected region.

18. The method of claim 1, wherein the medical image is or comprises a 3D functional image.

19. (canceled)

20. The method of claim 1, wherein the medical image is or comprises a positron emission tomography (PET) image and/or a single photon emission computed tomography (SPECT) image obtained following administration of an agent to the subject.

21. The method of claim 20, wherein the agent comprises a PSMA binding agent.

22. (canceled)

23. The method of claim 21, wherein the agent comprises ¹⁸F.

24. The method of claim 23, wherein the agent is or comprises [18F]DCFPyL.

25. The method of claim 21, wherein the agent is or comprises PSMA-11.

26. The method of claim 21, wherein the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

27. A method for interactive control of selection and/or analysis of regions of interest (ROIs) detected within a medical image of a subject and representing potential lesions, the method comprising:
(a) receiving and/or accessing, by a processor of a computing device, (i) an identification of a plurality of ROIs having been detected within the medical image, and, (ii) a set of ROI feature values comprising, for each particular ROI of the plurality of detected ROIs, corresponding value(s) of at least one ROI feature;
(b) causing, by the processor, display of a graphical control element allowing for user selection of a subset of the plurality of detected ROIs via user adjustment of one or more displayed indicator widgets within the graphical control element from which values of one or more user-selected criteria are received;
(c) determining, by the processor, based on a user adjustment of the one or more displayed indicator widgets, user selected values of the one or more criteria;
(d) selecting, by the processor, a user-selected subset of the plurality of detected ROIs based on (i) the set of ROI feature values and (ii) the user selected values of the one or more criteria; and
(e) storing and/or providing, by the processor, for display and/or further processing, an identification of the user-selected subset.

28-74. (canceled)

75. A system for interactive control of selection and/or analysis of hotspots detected within a medical image of a subject and representing potential lesions, the system comprising:
a processor of a computing device; and
memory having instructions stored thereon, wherein the instructions, when executed by the processor, cause the processor to:
(a) receive and/or access (i) an identification of a plurality of hotspots, and, (ii) a set of hotspot feature values comprising, for each particular hotspot of the plurality of detected hotspots, a corresponding value of at least one hotspot feature representing and/or indicative of a certainty or confidence in detection of the particular hotspot and/or a likelihood that the particular hotspot represents a true physical lesion within the subject;
(b) cause display of a graphical control element allowing for user selection of a subset of the plurality of detected hotspots via user adjustment of one or more displayed indicator widgets within the graphical control element from which values of one or more criteria are determined;
(c) determine, based on a user adjustment of the one or more displayed indicator widgets, user selected values of the one or more criteria;
(d) select a user-selected subset of the plurality of initial hotspots based on (i) the set of hotspot feature values and (ii) the user selected values of the one or more criteria; and
(e) store and/or provide, for display and/or further processing, an identification of the user-selected subset.

76-78. (canceled)

79. The method of claim 1, wherein the plurality of hotspots have been detected within the medical image using a machine learning model.
